(19) United States
(12) Patent Application Publication
(10) Pub. No.: US 2018/0374584 A1
(43) Pub. Date: Dec. 27, 2018

(54) Dynamic Presentation of Individualized and Populational Health Information and Treatment Solutions

(71) Applicant: Orb Health, Inc., Phoenix, AZ (US)

(72) Inventor: Paul Oran, Phoenix, AZ (US)

(21) Appl. No.: 16/106,804

(22) Filed: Aug. 21, 2018

Related U.S. Application Data
(63) Continuation of application No. 13/748,070, filed on Jan. 23, 2013, now abandoned.
(60) Provisional application No. 61/589,519, filed on Jan. 23, 2012.
(60) Provisional application No. 61/646,399, filed on May 14, 2012.

(51) Int. Cl. G16H 50/50; G06Q 50/22; G16H 15/00

(52) U.S. Cl. CPC G16H 50/50; Y02A 90/22; Y02A 90/26; G16H 15/00; G06Q 50/22

(57) ABSTRACT

The disclosure herein relates generally to the dynamic presentation of health information and treatment options. Methods and systems for automatically causing the display of a desired visual depiction of health information and treatment options are disclosed.

### CROSS REFERENCE

This application is a continuation of U.S. patent application Ser. No. 13/748,070, filed on Jan. 23, 2013, which claims the benefit of (i) U.S. Provisional Patent Application Ser. No. 61/589,519, filed on Jan. 23, 2012, and to (ii) U.S. Provisional Patent Application Ser. No. 61/646,399, filed on May 14, 2012, the contents of each of which are incorporated by reference herein in their entirety.

### COPYRIGHT NOTICE

Contained herein is material that is subject to copyright protection. The copyright owner has no objection to the reproduction of the patent disclosure by any person as it appears in the Patent and Trademark Office patent files or records, but otherwise reserves all rights to the copyright whatsoever.

### BACKGROUND

The recent proliferation of computing technologies has made it possible to collect, analyze, and present many different types of information for the benefit of users of such computing technologies. Moreover, computing technologies have become prevalent in a number of health-related technologies. For instance, computing technologies are increasingly used by patients, consumers, care providers, doctors, hospitals, retail health clinics, physician networks, and accountable care organizations, among other such individuals and entities, to analyze and review health information.

Nonetheless, typical techniques for presentation of personalized health information to users, such as patients, are often considered inconvenient, inefficient, and/or difficult to utilize.

### SUMMARY

Accordingly, there is a need for systems and methods that provide for convenient, efficient, and easily usable presentation of individualized and populational health information and treatment solutions. Disclosed herein are such systems and methods.

In one aspect, a first method involves: (a) receiving test data indicating laboratory-test information corresponding to at least one patient-test date; (b) receiving, via a user interface, (i) analysis-type data indicating an analysis type and (ii) patient-history data indicating a particular patient-test date; (c) determining a test result based on at least the (i) received test data, (ii) the analysis type data, and (iii) the patient-history data; and (d) causing a visual depiction of the test result relative to a visual depiction of a human body to be displayed on a graphical display.

In another aspect, a first non-transitory computer readable medium having instructions stored thereon is disclosed. The instructions include: (a) instructions for receiving test data indicating laboratory-test information corresponding to at least one patient-test date; (b) instructions for receiving, via a user interface, (i) analysis-type data indicating an analysis type and (ii) patient-history data indicating a particular patient-test date; (c) instructions for determining a test result based on at least the (i) received test data, (ii) the analysis type data, and (iii) the patient-history data; and (d) instructions for causing a visual depiction of the test result relative to a visual depiction of a human body to be displayed on a graphical display.

In another aspect, a first system that includes a processor, a non-transitory computer readable medium, and program instructions stored on the non-transitory computer readable medium is disclosed. The program instructions are executable by the processor to: (a) receive test data indicating laboratory-test information corresponding to at least one patient-test date; (b) receive, via a user interface, (i) analysis-type data indicating an analysis type and (ii) patient-history data indicating a particular patient-test date; (c) determine a test result based on at least the (i) received test data, (ii) the analysis type data, and (iii) the patient-history data; and (d) cause a visual depiction of the test result relative to a visual depiction of a human body to be displayed on a graphical display.

In another aspect, a second method involves: (a) determining, based on received user health data, (i) a set of health categories and (ii) for each health category in the set of health categories, one or more respective health category metrics corresponding to the health category; (b) selecting, based on the respective one or more health-category metrics corresponding to the health categories in the set of health categories, at least one health category for visual indication; (c) selecting, based on at least one of (i) the selected at least one health category and (ii) the respective one or more health-category metrics corresponding to the selected at least one health category, a visual indication of the selected at least one health category; and (d) causing the selected visual indication of the selected at least one health category to be displayed on a graphical display, where the visual indication of the selected at least one health category is displayed relative to a visual depiction of a human body.

In another aspect, a second non-transitory computer readable medium having instructions stored thereon is disclosed. The instructions include: (a) instructions for determining, based on received user health data, (i) a set of health categories and (ii) for each health category in the set of health categories, one or more respective health category metrics corresponding to the health category; (b) instructions for selecting, based on the respective one or more health-category metrics corresponding to the health categories in the set of health categories, at least one health category for visual indication; (c) instructions for selecting, based on at least one of (i) the selected at least one health category and (ii) the respective one or more health-category metrics corresponding to the selected at least one health category, a visual indication of the selected at least one health category; and (d) instructions for causing the selected visual indication of the selected at least one health category to be displayed on a graphical display, where the visual indication of the selected at least one health category is displayed relative to a visual depiction of a human body.

In another aspect, a second system that includes a processor, a non-transitory computer readable medium, and program instructions stored on the non-transitory computer readable medium is disclosed. The program instructions are executable by the processor to: (a) determine, based on received user health data, (i) a set of health categories and (ii) for each health category in the set of health categories, one or more respective health category metrics corresponding to the health category; (b) select, based on the respective one or more health-category metrics corresponding to the health categories in the set of health categories, at least one health category for visual indication; (c) select, based on at least one of (i) the selected at least one health category and (ii) the respective one or more health-category metrics corresponding to the selected at least one health category, a visual indication of the selected at least one health category; and (d) cause the selected visual indication of the selected at least one health category to be displayed on a graphical display, where the visual indication of the selected at least one health category is displayed relative to a visual depiction of a human body.

These as well as other aspects, advantages, and alternatives will become apparent to those of ordinary skill in the art by reading the following detailed description, with reference where appropriate to the accompanying drawings.

### BRIEF DESCRIPTION OF THE DRAWINGS

FIG. 1 shows a simplified block diagram of an example network.

FIG. 2 shows a simplified block diagram of an example network access device.

FIG. 3 shows a simplified block diagram of an example server.

FIG. 4 shows a flowchart of a first example method

FIG. 5 shows aspects of an example user interface.

FIG. 6 shows an example analysis algorithm.

FIGS. 7-24 show aspects of an example user interface.

FIG. 25 shows a flowchart of a second example method.

FIGS. 26-37 show aspects of an example user interface.

### DETAILED DESCRIPTION

In the following detailed description, reference is made to the accompanying figures, which form a part thereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the spirit or scope of the subject matter presented herein. It will be readily understood that aspects of the present disclosure, as generally described herein, and illustrated in the figures, can be arranged, substituted, combined, separated, and/or designed in a wide variety of different configurations, all of which are contemplated herein.

### 1. Example Architecture

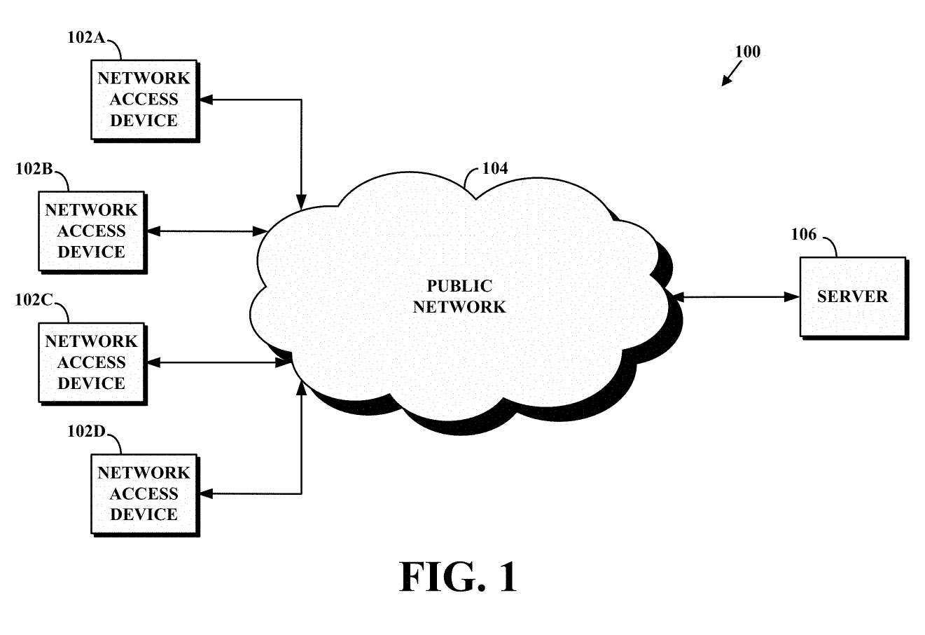

FIG. 1 shows a simplified block diagram of an example communication network in which the present method can be implemented. It should be understood that this and other arrangements described herein are set forth only as examples. Those skilled in the art will appreciate that other arrangements and elements (e.g., machines, interfaces, functions, orders, and groupings of functions, etc.) can be used instead and that some elements may be omitted altogether. Further, many of the elements described herein are functional entities that may be implemented as discrete or distributed components or in conjunction with other components, and in any suitable combination and location. Various functions described herein as being performed by one or more entities may be carried out by hardware, firmware, and/or software. And various functions described herein may be carried out by a processor executing instructions stored in memory.

As shown in FIG. 1, example network 100 includes various network-access devices 102A-102D, public network 104 such as the Internet, and server 106. Note that additional entities not depicted in FIG. 1 could be present as well. As an example, there could be more network-access devices and more servers in communication with public network 104. Other network elements may be in communication with public network 104 as well. Also, there could be one or more devices and/or networks making up at least part of one or more of the communication links depicted in FIG. 1. As an example, there could be one or more routers, switches, or other devices or networks on the communication links between network-access devices 102A-102D, public network 104, and/or server 106.

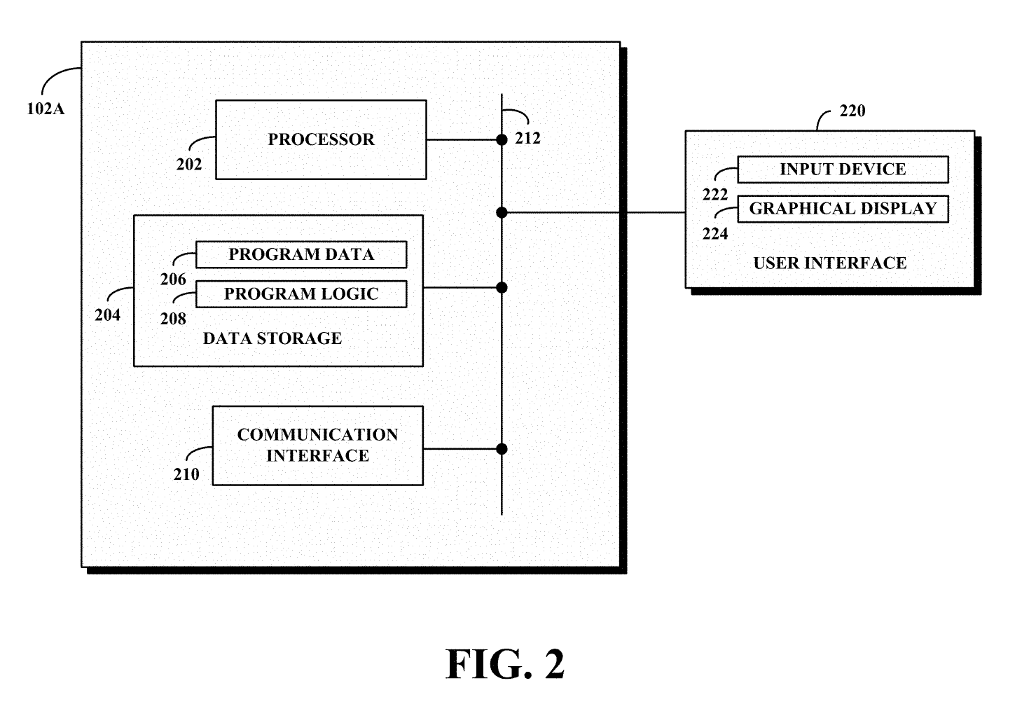

Each of network-access devices 102A-102D may be any network-access device arranged to carry out the network-access device functions described herein. Generally, the network-access device may be any suitable computing device, such as a computer, laptop computer, tablet computer, and/or smartphone, among other examples. The operation of the network-access device may be effected by software or firmware code stored in a non-volatile data store and executed via a general purpose processor transformed by the software or firmware code into a specific purpose processor, or may be effected solely by a hardware structure, or a combination of the two. As such each of network-access devices 102A-102D, including network-access device 102A as shown in FIG. 2, may include processor 202, data storage 204, and communication interface 210, all linked together via system bus, network, or other connection mechanism 212.

Processor 202 may comprise one or more general purpose microprocessors and/or one or more dedicated signal processors and may be integrated in whole or in part with communication interface 210. Data storage 204 may comprise memory and/or other storage components, such as optical, magnetic, organic or other memory disc storage, which can be volatile and/or non-volatile, internal and/or external, and integrated in whole or in part with processor 202. Data storage 204 may be arranged to contain (i) program data 206 and (ii) program logic 208. Although these components are described herein as separate data storage elements, the elements could just as well be physically integrated together or distributed in various other ways. For example, program data 206 may be maintained in data storage 204 separate from program logic 208, for easy updating and reference by program logic 208.

Communication interface 210 typically functions to communicatively couple network-access device 102A to networks, such as public network 104. As such, communication interface 210 may include a wired (e.g., Ethernet) and/or wireless (e.g., Wi-Fi) packet-data interface, for communicating with other devices, entities, and/or networks. Network-access device 102A may also include multiple interfaces 210, such as one through which network-access device 102A sends communication, and one through which network-access device 102A receives communication.

Network-access device 102A may also include, or may be otherwise communicatively coupled to, user interface 220. User interface 220 may include input device 222 comprising, for example, buttons, a touch screen, a microphone, and/or any other elements for receiving inputs. User interface 220 may also include one or more elements for communicating outputs, for example, one or more graphical displays 224 and/or a speaker. In operation, user interface 220 may be configured to display a graphical user interface (GUI) via graphical display 224 and may also be configured to receive inputs, via input device 222, corresponding to use of such a GUI.

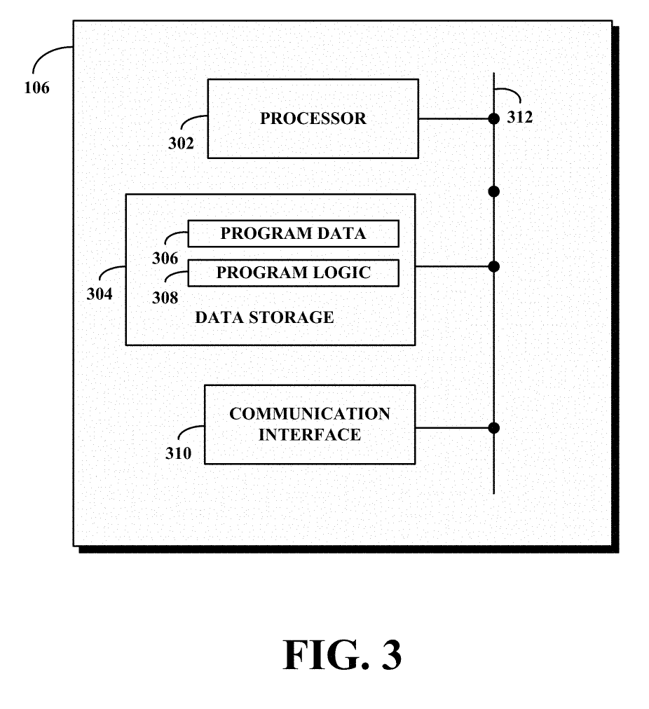

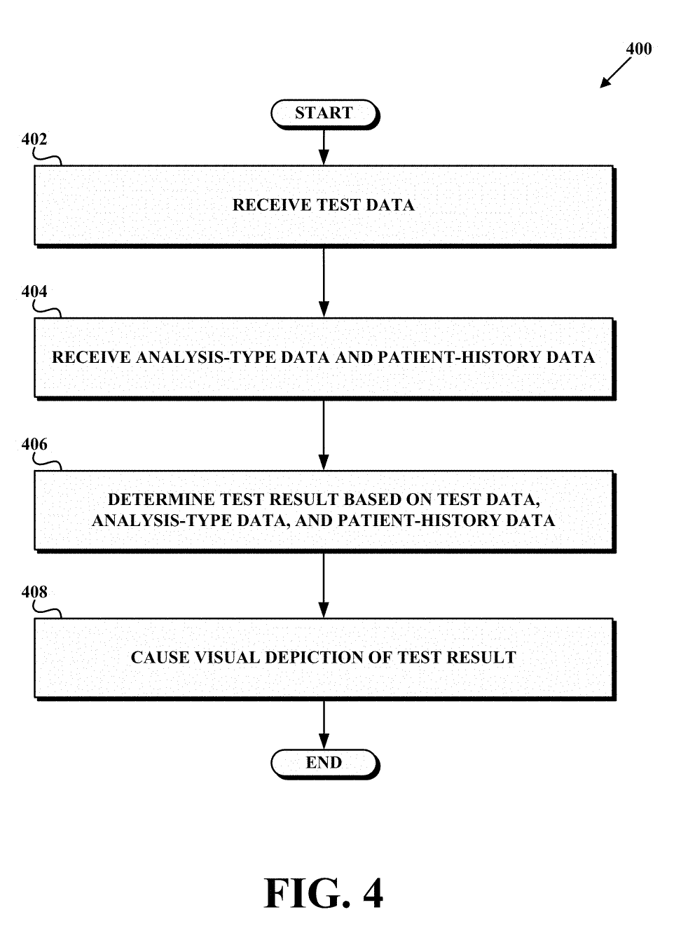

Server 106 may be any network server or other computing system arranged to carry out the server functions described herein including, but not limited to, those functions described with respect to FIG. 4. As such, as shown in FIG. 3, server 106 may include processor 302, data storage 304 comprising program data 306 and program logic 308, and communication interface 310, all linked together via system bus, network, or other connection mechanism 312. Processor 302, data storage 304, program data 306, program logic 308, and communication interface 310 may be configured and/or arranged similar to processor 202, data storage 204, program data 206, program logic 208, and communication interface 210, respectively, as described above with respect to network-access device 102A.

Data storage 304 may contain information used by server 106 in operation. For example, date storage 304 may comprise instructions executable by the processor for carrying out the server functions described herein including, but not limited to, those functions described below with respect to FIG. 4. As another example, data storage 304 may contain various design logic and/or design data used for determining a test result, such as the logic and data described below with respect to FIG. 4. Generally, data storage 304 may contain information used by server 106 to provide a graphical user interface that is accessible by various network-access devices, such as network-access device 102A, over public network 104.

Returning to FIG. 1, public network 104 may include one or more wide area networks, one or more local area networks, one or more public networks such as the Internet, one or more private networks, one or more wired networks, one or more wireless networks, and/or one or more networks of any other variety. Devices in communication with public network 104 (including, but not limited to, network-access devices 102A-102D and server 106) may exchange data using a packet-switched protocol such as IP, and may be identified by an address such as an IP address.

### 2. First Example Method

FIG. 4 shows a flowchart depicting an example method for dynamically presenting laboratory test results and treatment options. Method 400 is described, by way of example, as being carried out by a computing system such as, for example, server 106. However, it should be understood that example methods disclosed herein, such as method 400, may be carried out by computing systems other than a server, and/or may be carried out by sub-systems in a server or in other devices. For example, the example method may alternatively be carried out entirely by a network-access device or some other computing system that may or may not be coupled to any network. Other examples are also possible.

Furthermore, those skilled in the art will understand that the flowchart described herein with respect to FIG. 4 illustrates functionality and operation of certain implementations of example embodiments. In this regard, each block of the flowchart may represent a module, a segment, or a portion of program code, which includes one or more instructions executable by a processor (e.g., processor 302 described below with respect to server 106) for implementing specific logical functions or steps in the process. The program code may be stored on any type of computer readable medium (e.g., computer readable storage medium or non-transitory media, such as data storage 304 described above with respect to server 106), for example, such as a storage device including a disk or hard drive. In addition, each block may represent circuitry that is wired to perform the specific logical functions in the process. Alternative implementations are included within the scope of the example embodiments of the present application in which functions may be executed out of order from that shown or discussed, including substantially concurrent or in reverse order, depending on the functionality involved, as would be understood by those reasonably skilled in the art.

FIG. 4 shows a flowchart depicting functions that can be carried out in accordance with at least one embodiment of the method. As shown in FIG. 4 method 400 begins at block 402 with server 106 receiving (perhaps by way of public network 104), test data indicating laboratory-test information corresponding to at least one patient-test date. At block 404, server 106 receives, via user interface 220 of network-access device 102A (perhaps by way of public network 104), (i) analysis-type data indicating an analysis type and (ii) patient-history data indicating a particular patient-test date. At block 406, server 106 determines a test result based on at least the (i) received test data, (ii) the analysis-type data, and (iii) the patient-history data. At block 408, server 106 causes a visual depiction of the test result relative to a visual depiction of a human body to be displayed on graphical display 224 of user interface 220 of network-access device 102A. These steps are explained in the following subsections.

a. Receive Test Data

At block 402, server 106 receives (perhaps by way of public network 104), test data indicating laboratory-test information corresponding to at least one patient-test date. As discussed further below, the test data indicating laboratory-test information corresponding to at least one patient-test date may be used, in combination with analysis-type data and patient-history data, to determine and, ultimately, display to a user, a test result relative to a visual depiction of a human body.

The laboratory-test information corresponding to the at least one patient date as indicated by the test data may take any desired form. Generally, the laboratory-test information may reflect information concerning a laboratory test taken, executed, or otherwise performed on the at least one patient-test date indicated by the test data. For instance the laboratory-test information may include the results of an assortment of dozens or even thousands of uncategorized labs performed (genomics testing, in-vitro diagnostic testing) or even a single lab test (e.g. Glucose), the result from the lab test(s) which may contain qualitative or quantitative information (e.g. 85), the normal range or normal qualitative state of a healthy sample (e.g. 65-99), and/or the units for the specific lab test (e.g. mg/dL). The laboratory-test information may include other information such as the patient's address, the ordering physician, patient-demographic information, policy information, and/or any patient-risk information provided by the laboratory. Other examples of laboratory-test information may exist as well.

The laboratory-test information may be received by server 106 via a direct and/or indirect communication link with a computing system, server, or other network entity associated with a laboratory that performs the laboratory test. In this way, server 106 may receive the laboratory-test information from the laboratory that performs the laboratory test. As one specific example, server 106 may access laboratory-test information generated by, and made accessible by, a laboratory by way of an application program interface (API) provided by the laboratory or an associated entity. Server 106 may receive such laboratory-test information via an API as test data.

Alternatively, the laboratory-test information may be received by server 106 as a result of manual user input. For instance, server 106 may cause network-access device 102A to provide a prompt, form, or other data-input interface for a user to input laboratory-test information generated by and received from the laboratory that performs the laboratory test. For instance, a user of network-access device 102A may receive laboratory-test information for the laboratory in paper form (or another form), and may then submit some or all of such laboratory-test information to server 106 as test data. Other examples of laboratory-test information may exist as well.

As another alternative, the test-data may also include patient-submitted information containing subjective and/or objective information experienced, determined, or otherwise acquired by the user. For instance, server 106 may cause network-access device 102A to provide a prompt, form, or other data-input interface for a user to input patient-submitted information. Examples of subjective patient-submitted information may include the user's mood, whether the patient is “tired,” and/or whether the patient is feeling “well.” Examples of objective patient-submitted information may include the user's weight, what the user ate at a given time, and/or an amount of sleep the patient experienced on a given day. Other examples of subjective and/or objective patient-submitted information may exist as well.

b. Receive Analysis-Type Data and Patient-History Data

At block 404, server 106 receives, via user interface 220 of network-access device 102A (perhaps by way of public network 104), (i) analysis-type data indicating an analysis type and (ii) patient-history data indicating a particular patient-test date.

The data may be input by a user in any suitable manner. In an embodiment, server 106 may cause network-access device 102A to display a question, series of questions, a data-entry field, or a selectable button, among other possible examples, that prompt or otherwise allow the user to specify certain analysis-type data and/or patient-history data. Accordingly, server 106 may store logic arranged to prompt the user in such a manner. The input provided in response to the prompt may be input by the user using input device 222, and the input may be received by server 106 in the form analysis-type data and/or patient-history data.

The analysis type indicated by the analysis-type data may take on any desired form. Generally, the analysis-type data may reflect a medical analysis that has been previously performed with respect to laboratory-test information corresponding to a given patient-test date. For instance, the analysis type may be at least one of cardiovascular, thyroid, diabetes, adrenals, sex hormone, cellular health, Liver, CBC, CMP, Lipids, Cardiac, Kidney, Iron, Autoimmune, Clotting Factors, Female Hormone, Male Hormone, Cancer Screening, Bone Health, Urinalysis, and/or various genetics tests. Other examples of analysis types may exist as well.

The analysis type need not be limited to those commonly-recognized types, such as those described above. For example, the analysis type may incorporate tests that are a part of any one or more of the analysis types discussed above. Such an analysis type may be “standardized” and offered to a user consistently over time such that when the user engages in multiple analyses over time, the results will be comparable. In this way, the user may advantageously be able to observe changes across various laboratory-test information, over time.

The patient-test date indicated by the patient-history data may be one of a variety of dates corresponding to available laboratory-test information. Generally, the patient-test date may reflect a date that the patient submitted to a medical test and provided a medical sample (e.g., a blood test).

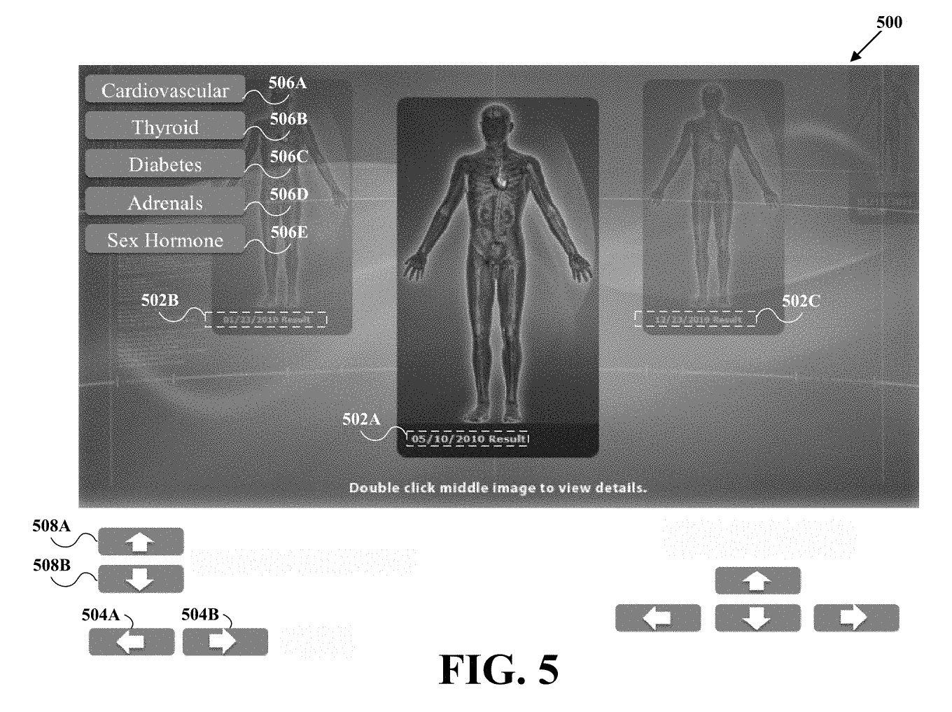

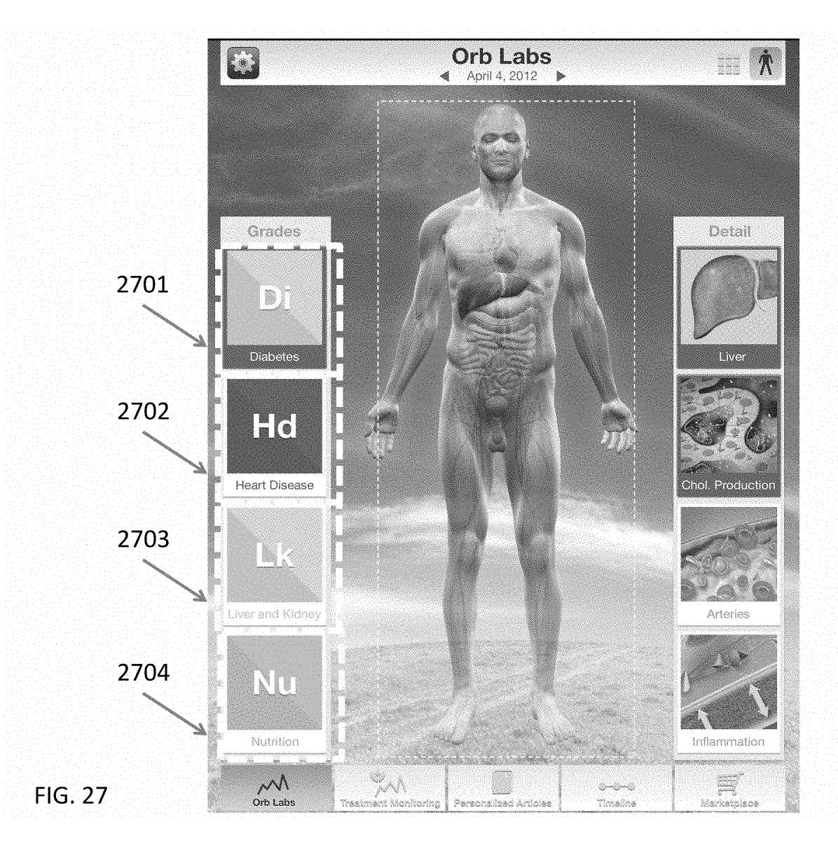

For purposes of example and explanation, FIG. 5 shows an example graphical user interface 500 capable of receiving user inputs. As shown, graphical user interface 500 includes a variety of analysis-type buttons 506A-506B, corresponding to cardiovascular and endocrine. The user may select any of the analysis-type buttons, such as cardiovascular, and thus provide the selected analysis type to server 106 as analysis-type data. FIG. 27 presents a simplified interface with similar functionality, where the user may select the results from their screenings and health/risk assessments for diabetes, heart disease, liver and kidney, and nutrition. Of note, and as previously mentioned, these categories are generated by automatically categorizing the lab tests associated with these particular conditions from raw patient data. As a particular example of receiving patient-test data, the user may be prompted to navigate, peruse, or otherwise browse through a variety of available patient-test dates. As shown, graphical user interface 500 includes a number of representations of the human body, each corresponding to various patient-test dates 502A, 502B, and 502C. Thus, the user may browse through the various patient-test dates by selecting any of patient-test dates 502A, 502B, and/or 502C (e.g., perhaps by selecting the patient-test date or human body associated with the respected patient-test date), and thus provide the selected patient-test date to server 106 as patient-history data.

Note that selection of a given patient-test date may cause that patient-test date, as well as the representation of the human body associated with the given patient-test date, to come to the front and center of graphical user interface 500. Thus, upon selection of patient-test date 502B, patient test date 502B may take the position of patient-test date 502A as shown in FIG. 5. Accordingly, patient-test date 502A may “shift” right and take the position of patient-test date 502C as shown in FIG. 5. Patient-test date 502C may then no longer be displayed.

As another example, of navigating, perusing, or otherwise browsing through a variety of available patient-test dates, graphical user interface 500 may include navigation buttons 504A and 504B. Upon selection of “left” navigation button 504A (i.e., by selecting left navigation button 504A), the user may browse to a patient-test date to the left (i.e., an earlier patient-test date) of the patient-test date that is currently front and center. Thus, in accordance with the example shown in FIG. 5, selection of left navigation button 504A may cause patient-test date 502B to take the position of patient-test date 502A.

Further, upon selection of “right” navigation button 504B (i.e., by selecting right navigation button 504B), the user may browse to a patient-test date to the right (i.e., a more recent patient-test date) of the patient-test date that is currently front and center. Thus, in accordance with the example shown in FIG. 5, selection of right navigation button 504B may cause patient-test date 502C to take the position of patient-test date 502A.

Further still, so as to call the attention of the user to the most critical information first, the visual depiction may show which laboratory-test information is out of range, or in a risky range, via a graphical depiction. Accordingly, such visual depictions of the laboratory-test information may fade in and out automatically such that the user may be alerted to the existence of, and ultimately view, additional test results. Such additional test results may be further explored by selecting the up and down arrows and cycling through all the available analysis types for more information.

c. Determine Test Result Based on Test Data, Analysis-Type Data, and Patient-History Data

At block 406, server 106 determines a test result based on at least the (i) received test data, (ii) the analysis-type data, and (iii) the patient-history data. Generally, server 106 may use the patient-history data received in accordance with block 404 to look up received-test data having the patient-test date indicated by the patient-history data. Server 106 may then use the analysis-type data received in accordance with block 404 to look up a given analysis corresponding to the given analysis type indicated by the analysis-type data. Server 106 may then retrieve a stored test result, perhaps from among a variety of test results, corresponding to the analysis type indicated by the analysis-type data. Thus, in one embodiment, server 106 may retrieve stored test result.

In another embodiment, determining the test result may involve server 106 selecting an analysis algorithm based on the analysis-type data. Server 106 may then apply the analysis algorithm to the received test data corresponding to the particular patent-test date to obtain at least one analysis result. Ultimately, server 106 may select, from the at least one analysis result, the test result.

The analysis algorithm used in accordance with block 406 may be, for example, any one of a variety of known medical algorithms. The term “medical algorithm” as used herein is meant to refer to any computation, formula, statistical survey, nomogram, or look-up table, useful in healthcare. Such medical algorithms may include decision tree approaches to healthcare treatment (i.e., if symptoms and/or lab test results A, B, and C are evident, then diagnosis X and treatment 1) and also less clear-cut tools aimed at reducing or defining uncertainty. Other examples of medical algorithms may exist as well.

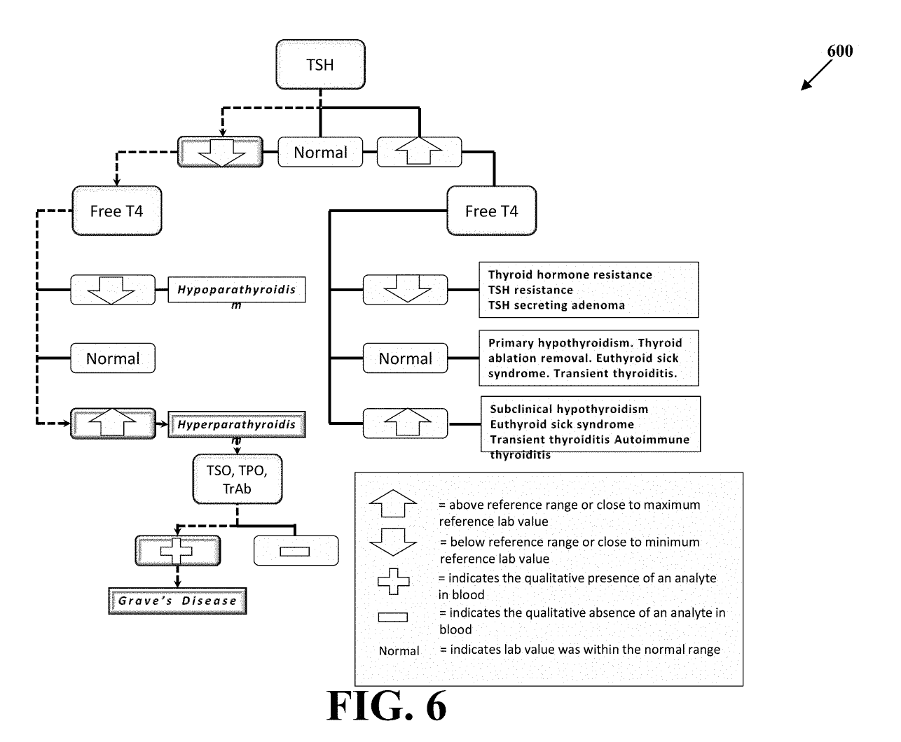

With reference to FIG. 6, a particular example of an analysis algorithm, in this case a known medical algorithm 600 is shown. Ultimately, server 106 may select, from at least one analysis result supplied by the analysis algorithm, a test result.

It should be noted that the particular medical algorithm 600 shown with respect to FIG. 6 is shown for purposes of example and explanation only, and should not be taken to be limiting. It should be understood that other analysis algorithms and/or medical algorithms may exist and that other such analysis algorithms and/or medical algorithms may be used in accordance with method 400 described herein. Furthermore, novel medical algorithms may also be utilized.

d. Cause Visual Depiction of Test Result

At block 408, server 106 causes a visual depiction of the test result relative to a visual depiction of a human body to be displayed on graphical display 224 of user interface 220 of network-access device 102A. Causing a visual depiction of the test result may involve causing a visual indication of the biological signatures of a disease or a state of good health. As one example, causing a visual depiction of the test result may involve visually highlighting or animating an organ. As another example, causing a visual depiction of the test result may involve highlighting or animating an organ system. The process of visually depicting a test result may involve dynamically rendering overlaid images or using 2D/3D graphic rendering.

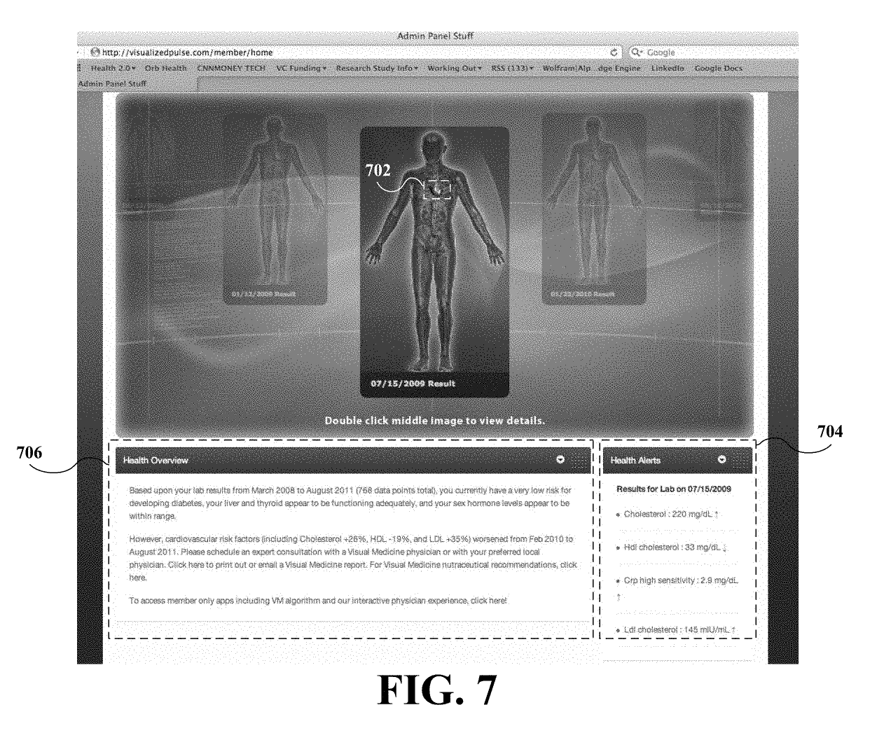

For instance, as shown with respect to FIG. 7, an organ such as the heart may by visually highlighted. In accordance with the example shown in FIG. 7, the test result determined in accordance with block 408 may have indicated a biological signature of a given disease implicating one or more issues with the heart. Thus, the heart may be highlighted to indicate one or more issues exist with respect to the test data associated with that patient-test date.

In this example, the following laboratory-test information may have caused the visual depiction of the heart to be visually highlighted: Cholesterol 220 mg/dL (which has a 100-199 mg/dL reference range), HDL cholesterol 33 mg/dL (which has a >39 mg/dL reference range) and LDL cholesterol 145 mg/dL (which has a 0-99 mg/dL reference range). Note that reference ranges can change depending upon the analytical platform. Furthermore, a graduated color scale depending on the severity of the test results can be applied. For example, a green color may indicate “health” (i.e., within range), a yellow color may indicate a “medium risk” (i.e., approaching range limit), and a red color could indicate “high risk” (i.e., outside of range). As noted above, the visual depiction of the test result may also, or alternatively, involve highlighting an organ system. Thus, as one example, to the extent the test result determined in accordance with block 408 may indicate a biological signature of a given disease implicating one or more issues with the cardiovascular system, the cardiovascular system as a whole may be highlighted (as opposed to the heart alone).

Aspects of FIG. 6 may also be implemented as a visual depiction, which may include multiple visualization possibilities—i.e. “visual algorithms.” Such “visual algorithms,” or visual depictions, may be embedded into a single so-called lab/blood panel (e.g. Thyroid “visual algorithm” panel) wherein several distinct biological outcomes related to the thyroid gland may be visualized using organs, organ systems, biochemical pathways, etc. For example, if test results were comprised of “low” TSH, “high” free T4, “high” T3, Positive TSI, Positive TPO, and Positive TRAb+, a possible diagnostic outcome may be a form of Hyperthyroidism or Graves disease. As such, the biological signatures of this disease may be visually depicted via a glowing and/or growing thyroid gland, animated eyes (i.e., one's eyes tend to bulge with graves disease), and/or a glowing and/or an accelerated beating heart animation (i.e. graves disease is often accompanied with an increased heart rate). As such, visual depictions of laboratory test data can comprise possible symptomatic or phenotypic expressions, biochemical pathways, late-stage disease symptoms, subjective information (e.g., emotional states), etc. Lastly, the regions of the body that are being visually depicted may be clicked on and interacted with so as to access more detailed diagnostic information.

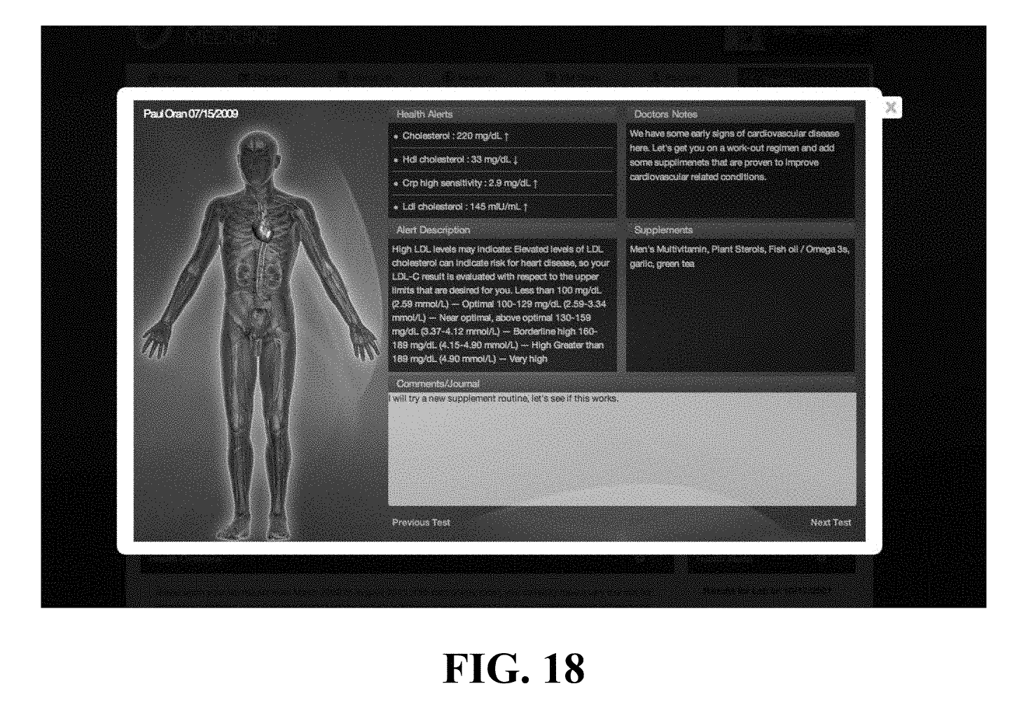

In addition to causing a visual depiction of the test result relative to a visual depiction of a human body, server 106 may also cause a visual depiction of additional information of interest related to the test result determined with respect to block 408. For example, various health alerts 704 may be displayed, which may be based on various determined test results or test data alone. In the particular example shown in FIG. 7, health results 704 include information such as a cholesterol level, an Hdl cholesterol level, a Crp high sensitivity, and a Ldl cholesterol level. In this way, the user may be provided a convenient overview of particular information of interest related to the test data. Also, as shown in FIG. 18, a window presenting more information (e.g., detailed test information, physician notes, test-drug interactions, supplements, journal functionality, etc.) may be displayed as a result of clicking in the interface somewhere (such as upon the visual depiction of the human body).

Further, server 106 may also cause a visual depiction of overview information corresponding to the test result determined with respect to block 408 and/or additional information accessible by server 106 (such as the test data). For example, a health overview 706 may be displayed. In the particular example shown in FIG. 7, health overview 706 includes information explaining information of interest compiled from test data obtained over a period of time (i.e., March 2008 to August 2011). In this way, the user may be provided a convenient overview of longitudinal health information of interest related to all, or some, of the test data received by server 106 regarding the user.

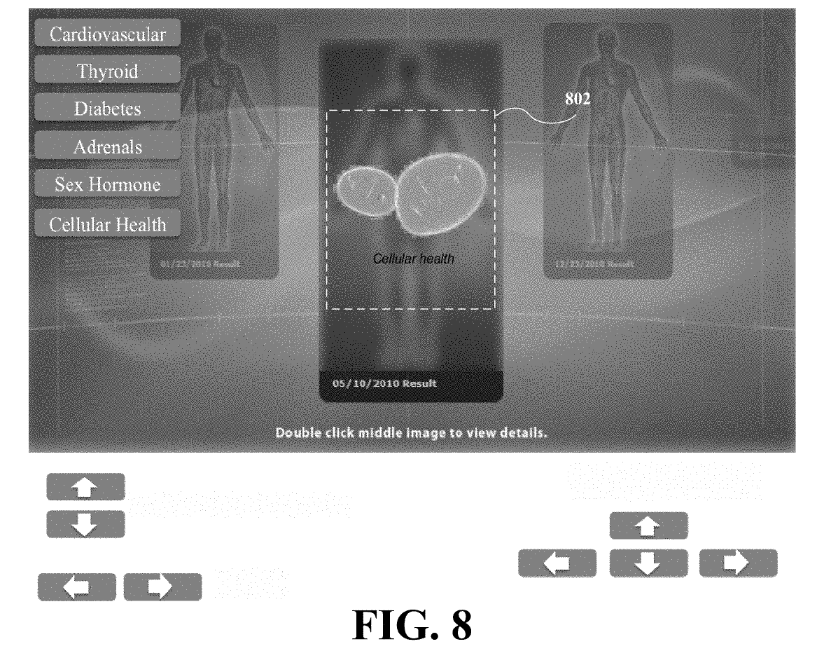

FIG. 8 shows another example of a visual depiction that may be provided. Cellular visual depiction 802, for instance, shows a visual depiction of cellular health. Such cellular health may be determined by server 106 based on a complete blood count (CBC) panel or other such analysis of test data. Cellular visual depiction 802 may display, for instance, a red cell distribution width (RDW), indicating the size of red blood cells. In an embodiment, the size of red blood cells may be shown relative to the size of healthy red blood cells. Also, or alternatively, in an embodiment, the size of red blood cells may be shown relative to a width scale (so as to indicate the absolute, or actual, width of the red blood cells).

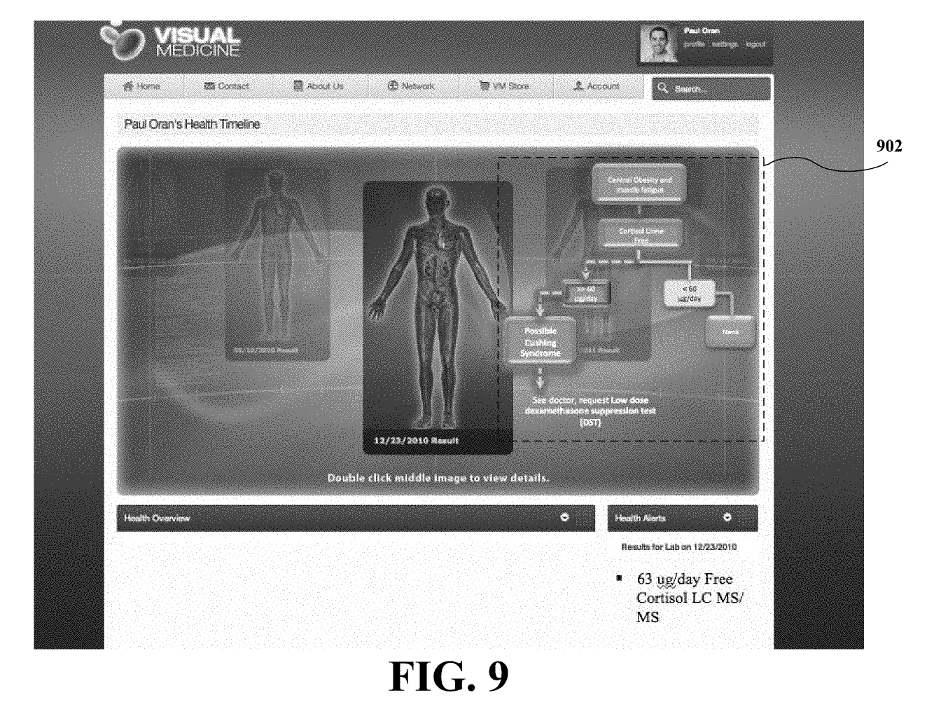

FIG. 9 shows another example of a visual depiction that may be provided. Medical-algorithm visual depiction 902, for instance, shows a visual depiction of a medical algorithm that may have been used to determine the test based on at least one of multiple sources of information including laboratory-test information, subjective patient-submitted information, and/or objective patient-submitted information as discussed above. In this way, the user may be informed of the analysis of the test data that has been performed. In an embodiment, medical-algorithm visual depiction 902 may be highlighted and/or animated so as to provide an indication to the user of the various paths of the medical algorithm that were followed to arrive at the test result.

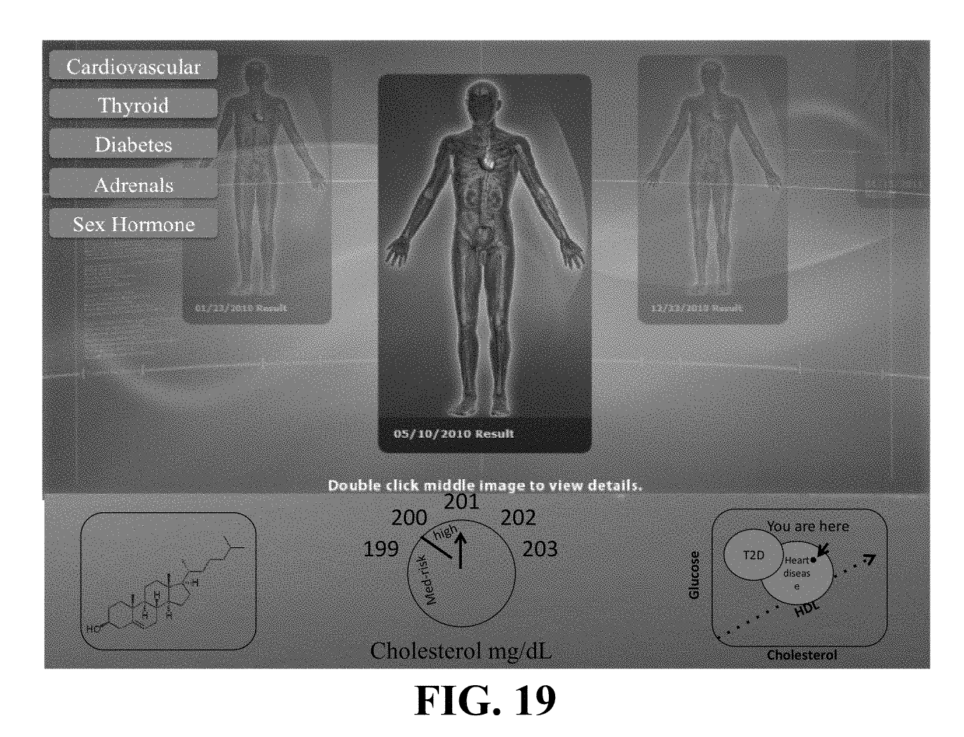

FIG. 19 depicts, inter alia, various “widgets” or “modules” that may be implemented so as to aid, support, or otherwise assist with an individual's comprehension of laboratory data with regards to several aspects including: the structure and composition of the analytes being tested, the various quantitative or qualitative test results using, for example, an animated “number-wheel” or “speedometer” GUI, and finally a display that provides populational information indicating one's degree of health relative to other individuals within a population.

Starting with the structure module, such a display may provide the viewer with compositional and/or structural information via Lewis structures, condensed formulas, skeletal formulas, perspective drawings, fischer projections, line or stick representations, electron-density plots, ball and stick models, space-filling modules, and/or cartoon representations, which may be rotated and/or interacted with. Further, a “speedometer” test-readout graphic may combine the visual human body interface with a quantitative test result in an easy to read and understand layout. In this context, while the visual depiction may highlight and animate the human body “displaying” the test result, the “speedometer” interface may “cycle” through the various quantitative and qualitative test results while the human body is currently displaying a specific/single piece of information. Further still, the “speedometer” may include speedometer arrow and numbers “spinning” as an animation as each lab test changes to get to the right number or phrase. The speedometer may also contain at-risk information embedded within the speedometer.

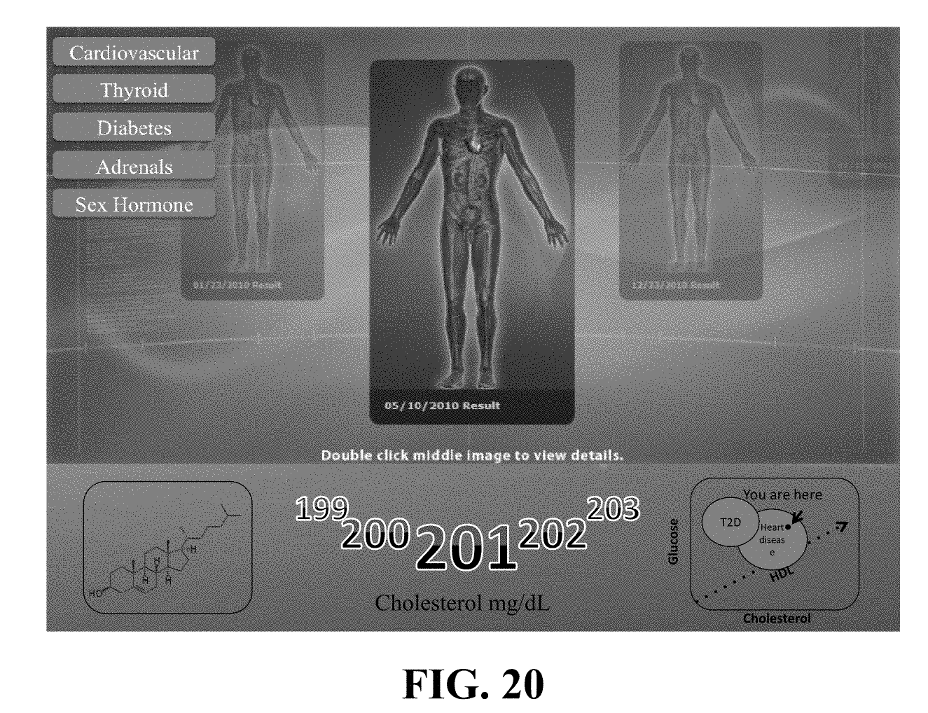

Additionally, and/or alternatively, as depicted in FIG. 20, an animated “number-wheel” graphical user interface may be implemented. As such, when the platform is isolated on a specific panel, the test's numbers or phrase may fade in and out, which may include the number wheel changing and/or spinning to get to the right number or phrase. Lastly, a populational-information “module” may be placed within the visual interface to aid, support, or otherwise assist with an individual's comprehension of how they compare to cohorts of healthy and/or sick individuals. A 2D or 3D plot for displaying how an individual compares to a population of healthy and/or sick individuals with regards to at least 2 diagnostic values or 2 panels of diagnostic tests within the platform can be graphically rendered as depicted in FIGS. 19 and 20. Each module may be added or removed from the default human body display according to, for example, the user's preference.

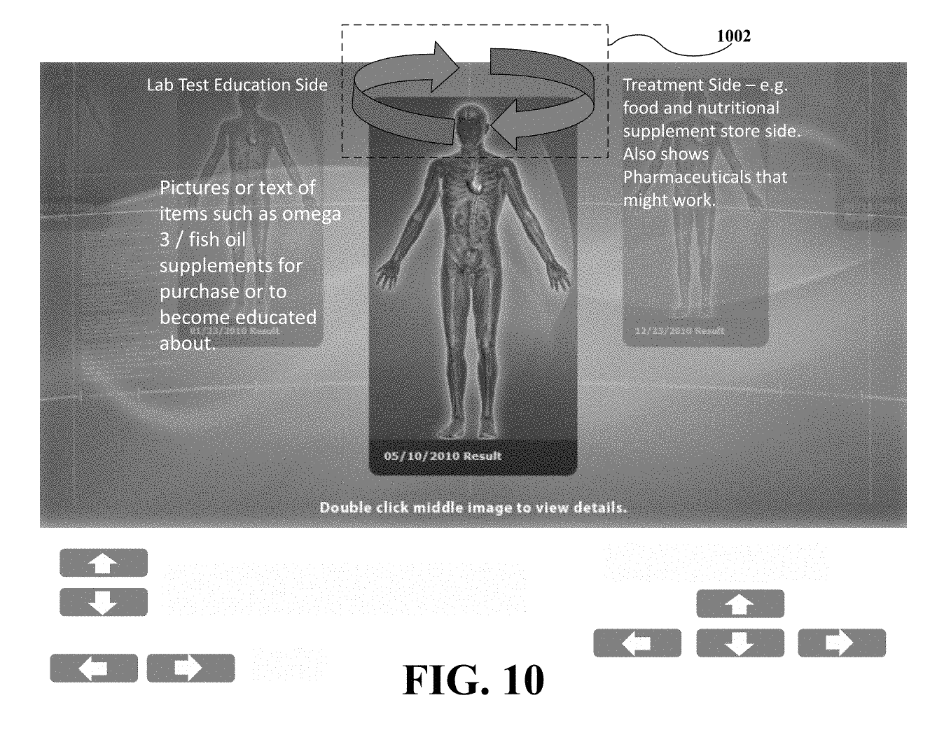

FIG. 10 shows another example of a visual depiction that may be provided. In accordance with the example shown in FIG. 10, the user may be capable of switching, or toggling, between various views of the representation of the human body. Different views of the human body may provide different types of information. For instance, a first view may provide diagnostic-education information related to laboratory-test history (e.g., information discussed with respect to FIGS. 6-9). A second view, however, may provide information concerning available treatment and/or preventative health goods such as food, nutritional supplements, and/or pharmaceuticals known to support treatment or prevention of a given condition. As will be discussed further below, options to purchase products related to treatment options may also be provided to the user.

e. Additional Functions

Server 106 may be configured to carry out various functions in addition to those functions described with respect to FIG. 4.

i. Purchase Functionality

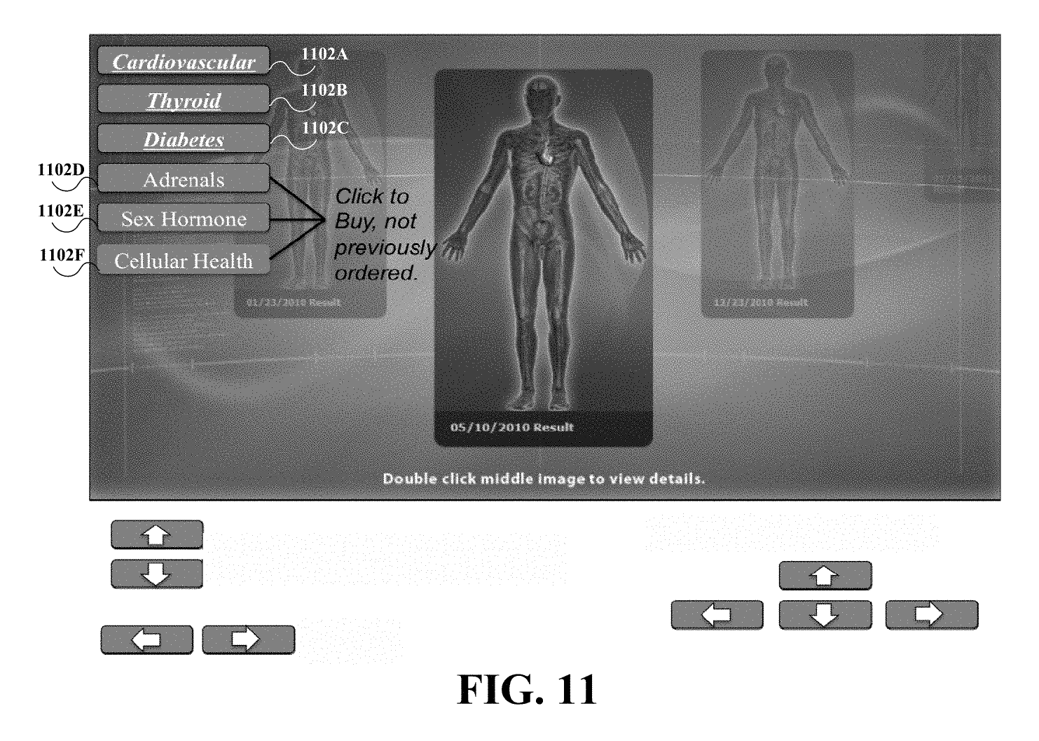

As one example, server 106 may determine at least one missing laboratory test result based on at least the received test data. Server 106 may then cause a visual depiction of the at least one missing laboratory test result to be displayed on a graphical display. For instance, with respect to FIG. 11, Cardiovascular button 1102A, Thyroid button 1102B, and Diabetes button 1102C are italicized and underlined, indicating that each such analysis has been performed on the test data associated with the selected patient-test date. However, Adrenals button 1102D, Sex Hormone button 1102E, and Cellular Health button 1102F are neither italicized nor underlined, indicating that such analyses have not been performed on the test data associated with the selected patient-test date. It should be understood that the example shown in FIG. 11 is shown for purposes of example and explanation only.

Accordingly, after causing the visual depiction of the test result relative to the visual depiction of the human body to be displayed, server 106 may be configured to receive, via the user interface, purchase data indicating a purchase of at least one of (a) a laboratory test and (b) a health-care product. Accordingly, server 106 may be configured to prompt the user, using suitable visual indications displayed via graphical display 224, to input, via input device 222, an indication that the user desires to purchase the analyses that have not yet been performed, such as an Adrenals, Sex Hormone, or Cellular Health analysis. Network-access node 102A may be configured to transmit to server 106 purchase data that indicates the user's desire to purchase a given analysis.

Further, server 106 may be configured to, after receiving the purchase data, transmit the received purchase data to an analysis lab. Generally, the purchase data may include information sufficient to enable the analysis lab to perform the requested analysis. In this way, a request for a given analysis may be submitted to an analysis lab upon receipt of the user's purchase.

ii. Populational Information Visualization and Social-Network Functionality

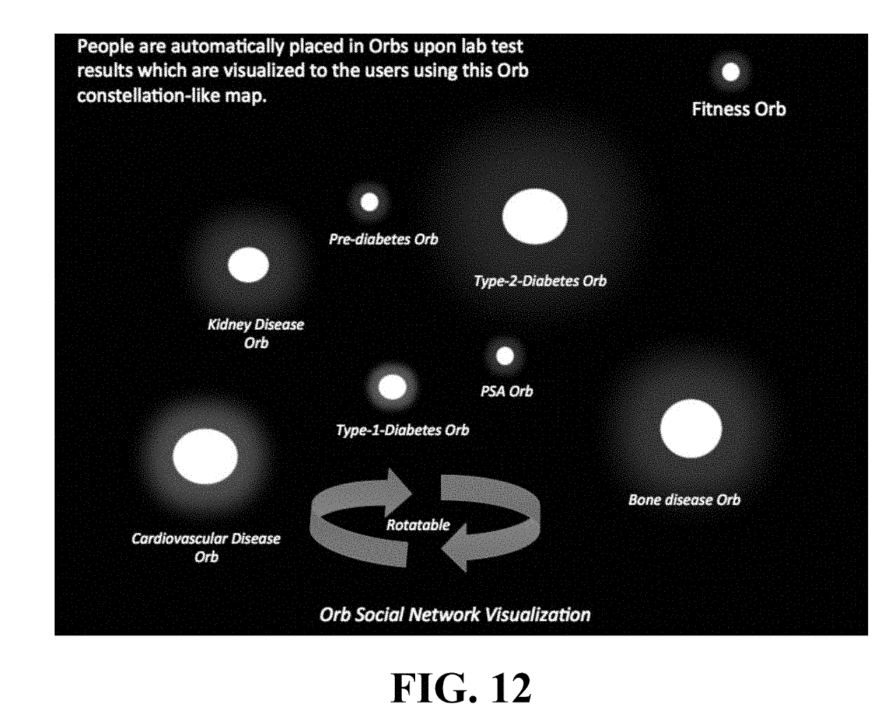

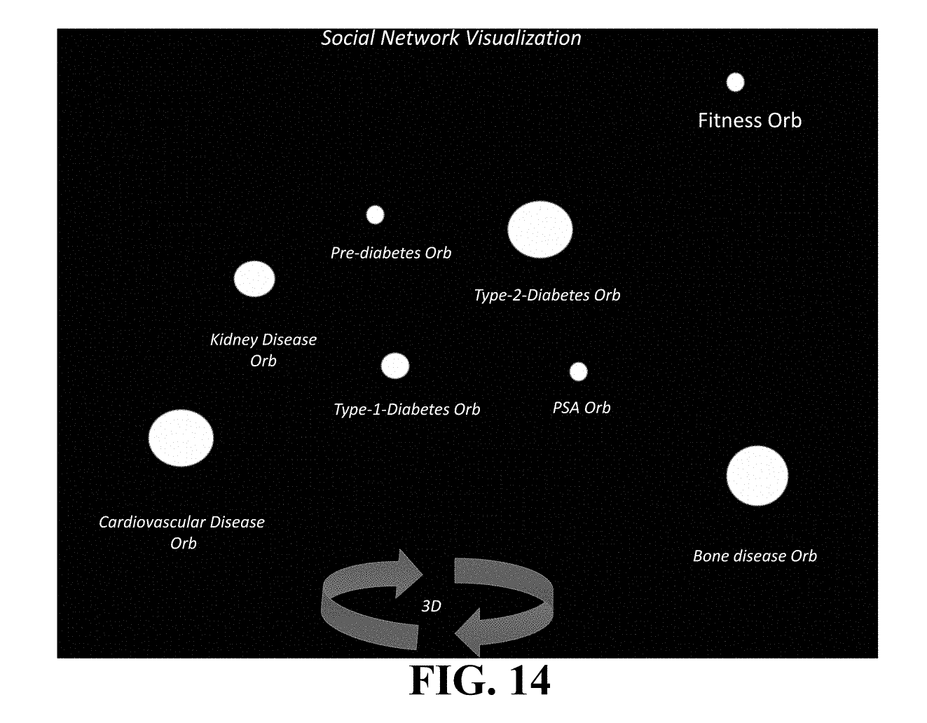

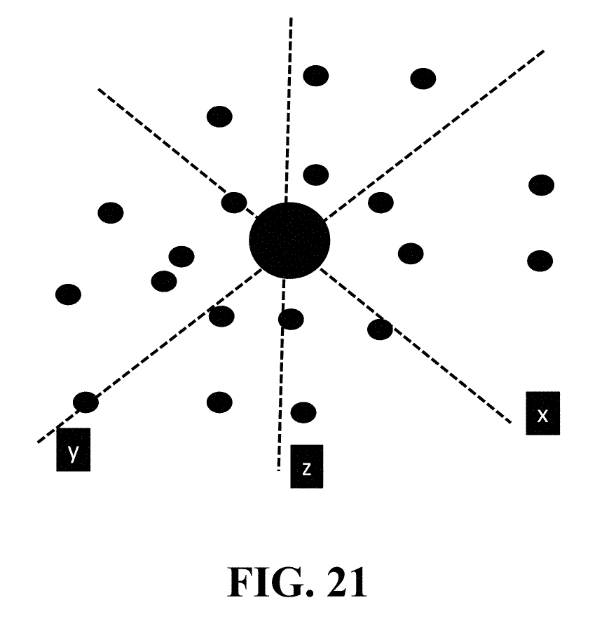

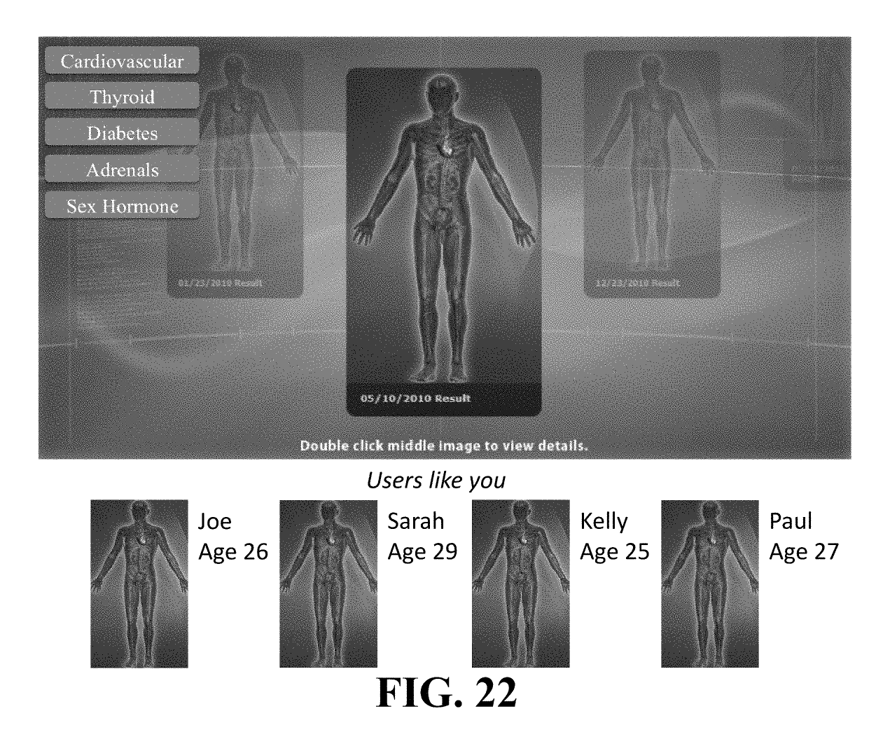

Users may be placed into social groups using computational algorithms that take into account diagnostic information (e.g. laboratory information), objective patient-submitted information, and/or subjective patient-submitted information from each respective user. The resulting social network may then include groups of users. Example graphical user interface that combine a visual depiction of diagnostic data with finding others are presented in FIGS. 12, 14, and 21. In this example, other individual's visual depictions may be displayed so as to provide an easy means of communication and assessment of relation to other users, as depicted in FIG. 22.

Collectively, users in the social network may be visualized using a graphical user interface that displays the various social groups that may be formed based on medical diagnostic information (e.g. laboratory test information), objective patient-submitted information, and/or subjective patient-submitted information from each respective user. More particularly, the graphical user interface may include a “constellation” or “galaxy” presentation.

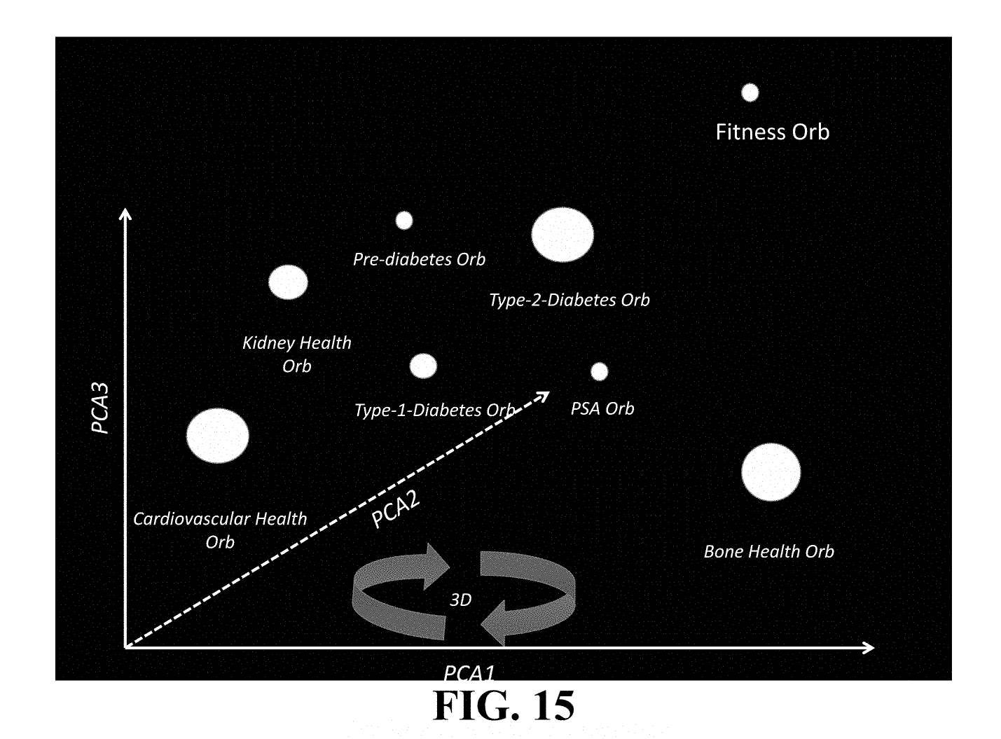

Such a constellation or galaxy presentation may be interactive and may be rotated, zoomed in and out, manipulated, and/or re-organized into three dimensions depending on, for example, user input. Further, the social network may be sorted into discrete and/or well-defined groups using clinical evidence and computer algorithms. Such algorithms may sort the user into a given social group. Such a constellation presentation is represented in FIG. 15.

Alternatively, data associated with all or a portion of the users of the social network may be evaluated in addition to, or in combination with, the functions described with respect to FIG. 4 using, for example, unsupervised and/or supervised multidimensional procedures. Such procedures may “auto-sort” the population into specific groups. For example, variable normalized principal component analysis (PCA) can be applied to the entire data set (i.e., data associated with all or a portion of the users of the social network which may include laboratory diagnostic information and objective and subjective health information).

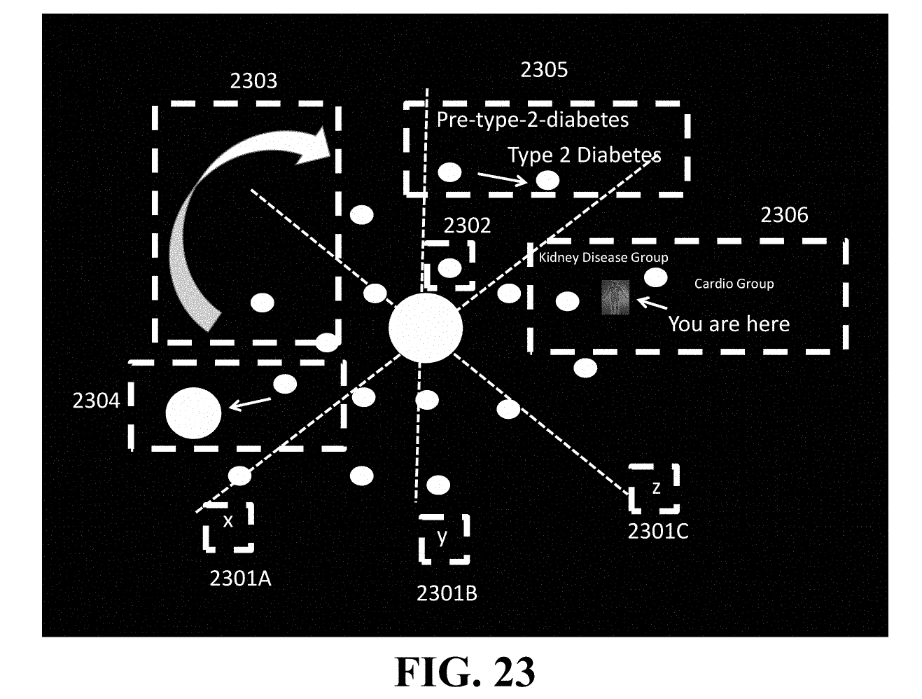

Accordingly, as a non-limiting example, thousands of users and millions of data points may reveal where “groups” of individuals exist. Such groups may then be visualized using a 3D plot according to three relevant PC scores. Such a visualization may make a visual representation of the social network dynamic and evolving—that is, the visual representation may change over time depending on the users of the social network. Individual principal component scores may be influenced by factors such as glycemic relationships (e.g. glucose levels and HBA1C), cellular health (e.g. CBC), and protein post-translational modifications (e.g. truncation), among many others. Thus, a 3D visual of three principal component scores (via plotting on x, y, and z axes as depicted in FIG. 23, axes 2301A, 2301B, and 2301C, respectively) may yield disease-stratified, health-stratified, treatment-stratified, and/or fitness-stratified cohorts and/or social groups which can be overlaid into a graphic environment such as a galaxy or constellation like presentation. Other statistical procedures can also be used.

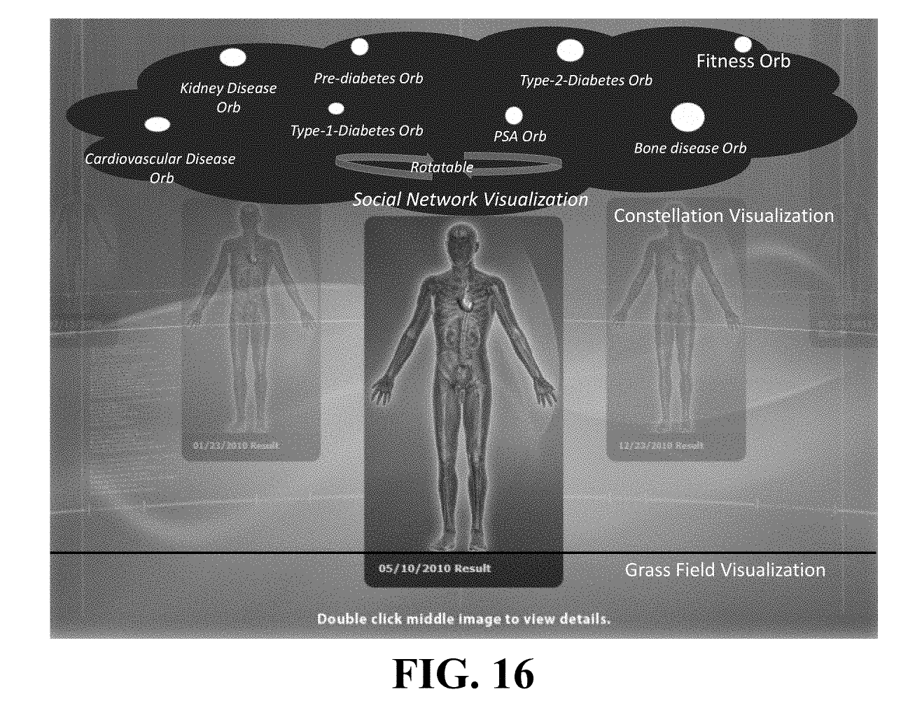

Further still, the resulting social-network visualization may be integrated into the laboratory test visualizer platform (i.e., a human body timeline) as a “constellation” or “galaxy” presentation above a visual depiction of the human body, so as to appear to exist in the sky within the graphical user interface. The various social groups the user belongs to may also be highlighted above the human body and/or “focused” upon. Such a constellation presentation above the human body is represented in FIG. 16.

And further still, the network of social groups (2302) can be joined by clicking on, using keyboard arrows, and/or touching via a multi-touch interface, among other examples. The “galaxy” can exhibit movement and or rotation (2303). Such motion may be computed by treating the relative size of social group, and/or relative severity of the disease, as-if it has similar properties as gravity (i.e., larger social groups may attract smaller groups—2304). Furthermore, motion of various social groups may have “clinical” trajectories in space/time (2305). These various “trajectories” may be determined by various predictive clinical outcomes using available literature, clinical studies, and dynamic evidence created from the platform itself. In other words, if an individual or group of individuals is in a particular group in space with a trajectory or “orbiting” and a larger body, a predicted path may be presented to the viewer as a means to let them know their predicted eventual state of health if they don't change lifestyle, treatments, etc. Furthermore, “satellite-groups” may also be displayed which provide more-specific groups of individuals that are exhibiting co-morbidities of broader diseases. Such broader diseases may be presented by the parent/larger object nearby.

And further still, a statistical calculation and representation of health and disease can be utilized to help sort new individuals into specific social groups (2306). For example, if an individual's diagnostic information yields precise “coordinates” in space, the user will be placed automatically in the “nearest” social groups or offered to join such nearest social groups. In FIG. 23, for example, the individual may be placed in both the kidney disease and cardio social group.

And further still, the social-network visualizations, and/or information corresponding thereto, may be integrated into other social-network platforms. For instance, a user of the social network described herein may “share” visualization (e.g. personal information such as the human body or galaxy) with a third-party social network such that the third-party-social network may display the visualization. Alternatively a user of the social network described herein may “share” laboratory-test information with a third-party social network such that the third-party social network may display the laboratory-test information (i.e., as a “status update,” among other examples).

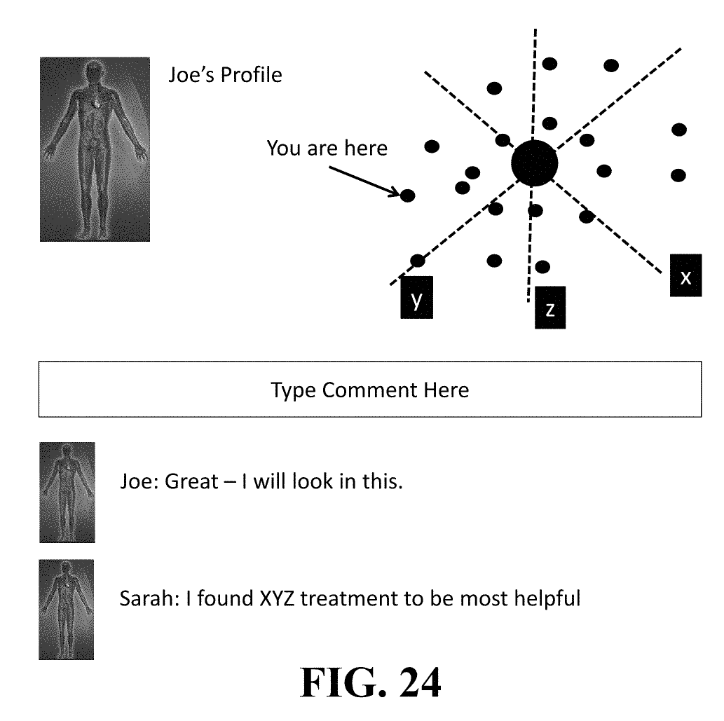

And, as depicted in FIG. 24, the galaxy or constellation visual can be embedded into a social group dialog so as to provide a reference to where (i.e. in relation to space/time in the galaxy visual) and what (i.e. the clinical definition—diabetes, pre-diabetes, etc) the user is associated with.

iii. Alternative Laboratory-Test Result Access

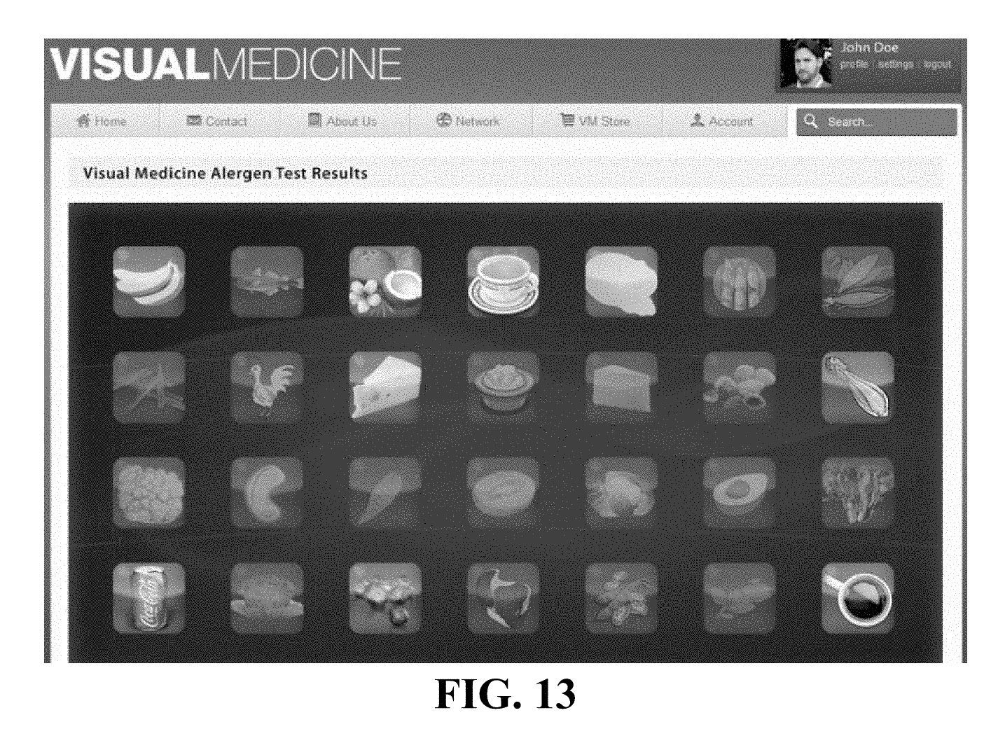

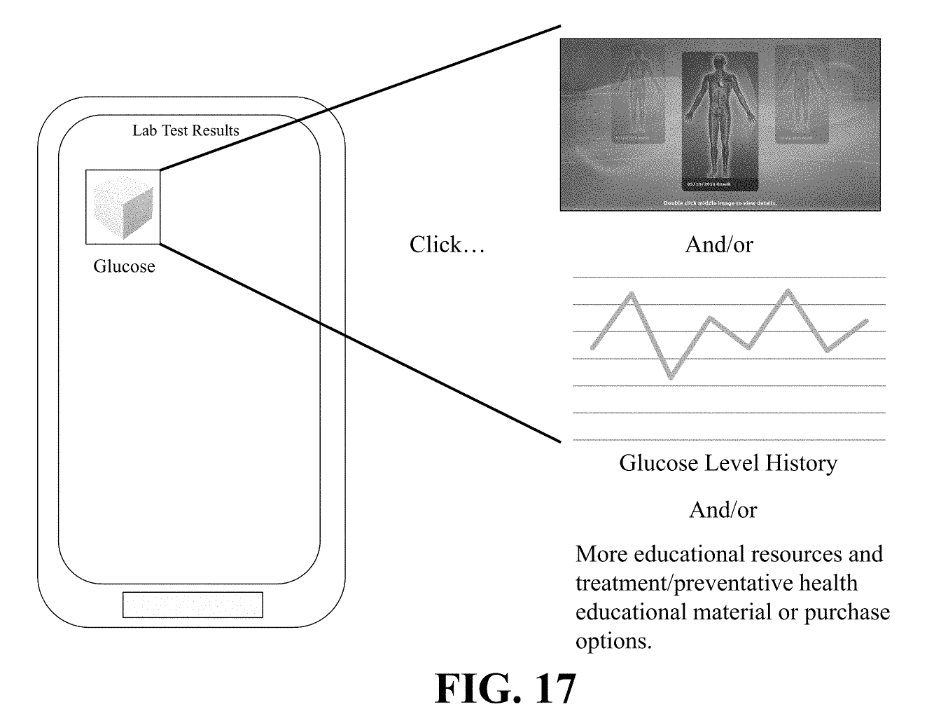

An alternative technique for displaying-laboratory test results may involve a graphical user interface including a number of graphical icons, where laboratory-test results are displayed as graphical icons that may be selected. Such a user interface may resemble the user interface depicted in FIG. 13. In a particular embodiment, such a user interface may resemble the user interface and/or functions depicted in FIG. 17.

More particularly, with respect to FIG. 17, test results for glucose may be displayed as a “sugar-cube” graphical icon. The user may then select the graphical icon (i.e., the “glucose” graphical icon) to explore more information, such as the laboratory history for glucose levels, its relationship to the body (e.g., using a human body interface), and possible treatment and preventative health options (e.g., food options that have a low glycemic index).

With respect to FIG. 13, for a particular example of displaying test results for individuals that have had an allergen panel run (e.g. a comprehensive test to determine which foods one is allergic to), an interactive “food button” menu may display test results by de-emphasizing items the user is not allergic to (i.e., graying out, blurring, etc.) and emphasizing items the user is allergic too (i.e., colored, animated, etc.). In this way, allergens may be displayed visually to provide a beneficial viewing experience.

Furthermore, such food buttons may be interactive. In other words, each button may serve as both a test result and as a shortcut that launches an application. Upon selecting a food button, information including food substitutes, health information, lifestyle change recommendations, etc. may be displayed so as to provide a more rich experience for viewing allergen test results.

### 3. Second Example Method

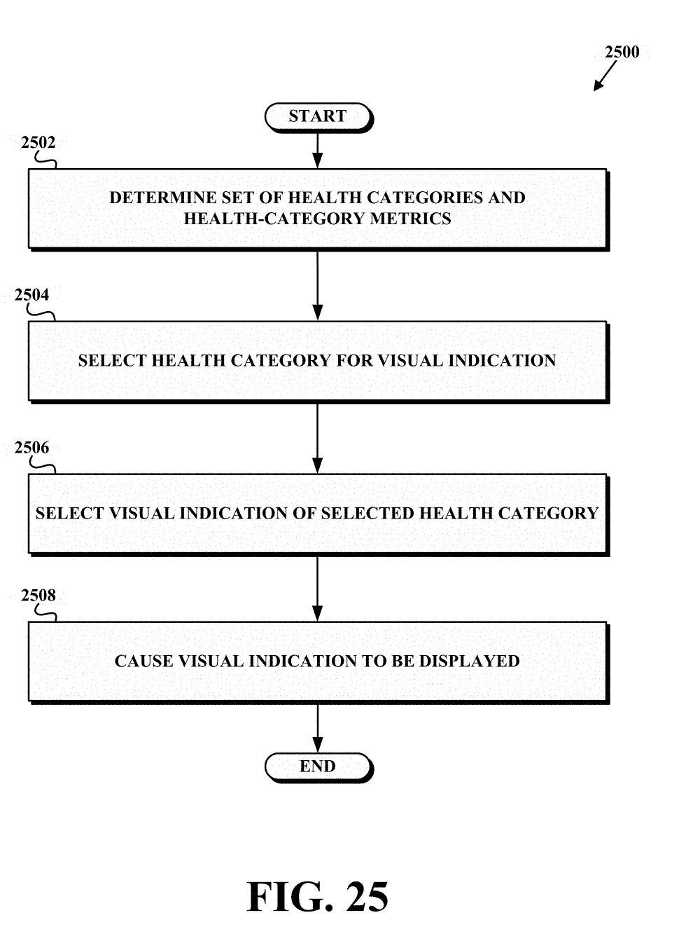

FIG. 25 shows a flowchart depicting an example method 2500 for dynamically presenting health information and treatment options. Method 2500 is described, by way of example, as being carried out by a computing system such as, for example, server 106. However, it should be understood that example methods disclosed herein, such as method 2500, may be carried out by computing systems other than a server, and/or may be carried out by sub-systems in a server or in other devices. For example, the example method may alternatively be carried out entirely by a network-access device or some other computing system that may or may not be coupled to any network. Other examples are also possible.

Further, those skilled in the art will understand that the flowchart described herein with respect to FIG. 25 illustrates functionality and operation of certain implementations of example embodiments. In this regard, each block of the flowchart may represent a module, a segment, or a portion of program code, which includes one or more instructions executable by a processor (e.g., processor 302 described below with respect to server 106) for implementing specific logical functions or blocks in the process. The program code may be stored on any type of computer readable medium (e.g., computer readable storage medium or non-transitory media, such as data storage 304 described above with respect to server 106), for example, such as a storage device including a disk or hard drive. In addition, each block may represent circuitry that is wired to perform the specific logical functions in the process. Alternative implementations are included within the scope of the example embodiments of the present application in which functions may be executed out of order from that shown or discussed, including substantially concurrent or in reverse order, depending on the functionality involved, as would be understood by those reasonably skilled in the art.

Further still, it should be understood that method 2500, and aspects thereof, may be carried out in addition to, in combination with, or instead of method 400 and aspects thereof. Indeed, as discussed further below, at least some of the data and/or functions described with respect to method 400 may be the same as or similar to data and/or functions described with respect to method 2500.

FIG. 25 shows a flowchart depicting functions that can be carried out in accordance with at least one embodiment of the method. As shown in FIG. 25 method 2500 begins at block 2502 with server 106 determining, based on received user health data, (i) a set of health categories and (ii) for each health category in the set of health categories, one or more respective health category metrics corresponding to the health category. At block 2504, server 106 selects, based on the respective one or more health-category metrics corresponding to the health categories in the set of health categories, at least one health category for visual indication. At block 2506, server 106 selects, based on at least one of (i) the selected at least one health category and (ii) the respective one or more health-category metrics corresponding to the selected at least one health category, a visual indication of the selected at least one health category. At block 2508, server 106 causes the selected visual indication of the selected at least one health category to be displayed on a graphical display, where the visual indication of the selected at least one health category is displayed relative to a visual depiction of a human body. These blocks are explained in the following subsections.

a. Determine Set of Health Categories and Health-Category Metrics

At block 2502, server 106 determines, based on received user health data, (i) a set of health categories and (ii) for each health category in the set of health categories, one or more respective health category metrics corresponding to the health category.

Health categories may be determined based on health data including, for example, in-vitro diagnostic testing, genetic testing, and/or other patient-submitted health data, among other examples. In an embodiment, the user health data may be laboratory test data. For purposes of example and explanation, health data may include individual lab test data (e.g., glucose or PSA), a series of uncategorized lab test data, or commonly used medical categories, yet still medically undefined/undiagnosed from collective data, including a complete blood count (WBC, RBC, Hemoglobin, Hematocrit, etc.) and lipid panel (cholesterol, triglyceride, HDL cholesterol, ldl cholesterol), among other examples. Such health data may be provided to the system in any suitable manner (such as submission by a user via an interface).

For purposes of example and explanation, a health category may be, or may indicate, an individual's state of health, status or diagnosis of disease, a prognosis or risk assessment, or a therapeutic response to a drug, among other examples. Alternatively or additionally, health categories may include family history, health interests, and genetics, among other examples. Some specific non-limiting examples of health categories include the presence (or lack of presence) of diabetes (screening/diagnosis), cardiovascular disease (risk assessment), liver and kidney (e.g. health/disease assessment), and nutrition (health/disease assessment).

Additional example health categories corresponding to diagnostics include, but are not limited to: Acid-Base Disorders, Acidosis and Alkalosis, Acidosis/Alkalosis, aCL Syndrome, ACS, Acute DIC, Acute inflammatory demyelinating polyneuropathy, Acute Myocardial Infarct, Addison's Disease, Adrenal Insufficiency, Adrenal Insufficiency & Addison's Disease, Albuminuria, Alcohol dependence, Alcoholism, Allergies, Alzheimer's Disease, AMI, Anemia, Anencephaly, Angiitis, Angina, Angina pectoris, Ankylosing Spondylitis, Anthrax, Anticardiolipin Antibody Syndrome, Antiphospholipid Antibody Syndrome, Antiphospholipid Syndrome, aPL Syndrome, APLS, APS, Arteritis, Arthritis, AS, Asthma, Atypical Mycobacteria, Atypical Pneumonia, Autoimmune Disorders, Autoimmune thyroiditis, Avian flu, Bacillus anthracis infection, Benign Prostatic Hyperplasia, Benign Prostatic Hypertrophy, Biological Warfare, Bioterrorism Agents, Bleeding Disorders, Bone Marrow Disorders, BPH, Breast Cancer, Cardiovascular Disease, Celiac Disease, Celiac Sprue, Cervical Cancer, CFIDS, CFS, CHF, Chlamydia, Chronic Fatigue and Immune Dysfunction Syndrome, Chronic Fatigue Syndrome, Chronic thyroiditis, Cobalamin, Colon Cancer, Colorectal Cancer, Community-Acquired Pneumonia, Congestive Heart Failure, Conn's Syndrome, Consumption, Consumption Coagulopathy, Copper storage disease, CREST, Cushing's Syndrome, Cutaneous anthrax, CVD, Cystic Fibrosis, Degenerative Joint Disease, Diabetes, Diabetes mellitus, Diarrhea, DIC, Diffuse Cutaneous Scleroderma, Diffuse thyrotoxic goiter, Disseminated Intravascular Coagulation, Disseminated Intravascular Coagulopathy, Disseminated lupus erythematosus, DJD, Double pneumonia, Down Syndrome, DS, Dysmetabolic Syndrome, Encephalitis, Endocrine Syndromes, Epilepsy, Excessive Clotting Disorders, Extraosseous plasmacytoma, Fibromyalgia, Flu, Folate Deficiency, Folic Acid, Fungal Infections, Gluten-Sensitive Enteropathy, Gonorrhea, Gout, Gouty arthritis, Graves Disease, Guillain-Barre Syndrome, H1N1, Hashimoto Thyroiditis, HD, Healthcare-Associated Pneumonia, Heart Attack, Heart Attack and Acute Coronary Syndrome, Heart Disease, Hemochromatosis, Hepatitis, Hepatolenticular degeneration, Herpes, Herpes Zoster, High blood pressure, HIV, HL, Hodgkin Lymphoma, Hodgkin's Disease, Hospital-Acquired Pneumonia, HPV, Hughes Syndrome, Human Immunodeficiency Virus, Huntington Disease, Huntington's Chorea Disease, Hypercoagulable Disorders or States, Hypersensitivity, Hypertension, Hyperthyroidism, Hypothyroidism, Infectious Arthritis, Infectious polyneuritis, Infertility, Inflammatory Bowel Disease, Influenza, Influenza A, Influenza B, Inhalation anthrax, Inherited copper toxicity, Insulin Resistance, Insulin Resistance Syndrome, Jaundice, JCA, JIA, JRA, Juvenile chronic arthritis, Juvenile Idiopathic Arthritis, Juvenile Rheumatoid Arthritis, Keratoconjuntivitis sicca, Kidney and Urinary Tract Function, Kidney Disease, Landry's ascending paralysis, LE, Lead Poisoning, Leukemia, Limited Cutaneous Scleroderma, Linear Scleroderma, Liver Disease, Lobar pneumonia, Localized Scleroderma, Lower Respiratory Tract Infection, Lung Diseases, Lupus, Lupus Anticoagulant Syndrome, Lupus erythematosus, Lyme Disease, Lymphoma, Malabsorption, Malaria, Malnutrition, Meningitis and Encephalitis, Menopause, Metabolic Syndrome, MI, Morphea, Multiple Myeloma, Multiple Sclerosis, Mycobacteria other than tuberculosis, Myelocele, Myelomeningocele, Myeloproliferative Disorders, Myocardial Infarct, Neural Tube Defects, Neuropathy, non-Hodgkin lymphoma, Nontuberculous Mycobacteria, Obesity Syndrome, Osteoarthritis, Osteoarthrosis, Osteoporosis, Ovarian Cancer, Pancreatic Cancer, Pancreatic Diseases, Pancreatic Insufficiency, Pancreatitis, PCOS, Pelvic Inflammatory Disease, Peptic Ulcer, Pituitary Disorders, Plasma cell dyscrasia, Plasma cell myeloma, Plasma cell neoplasm, Plasmacytoma, Plasmacytoma of bone, Pneumonia, Polycystic Ovarian Syndrome, Post-infectious arthritis, Pregnancy, Primary hyperaldosteronism, Prostate Cancer, Protein in urine, Proteinuria, Reactive Arthritis, Reaven Syndrome, Rheumatoid Arthritis, Rheumatoid spondylitis, Sarcoidosis, Scleroderma, Sepsis, Septic Arthritis, Sexually Transmitted Diseases, Sexually transmitted infections, Shingles, Sicca syndrome, Sickle Cell Anemia, Sickle Cell Disease, Sjögren Syndrome, Spina bifida, Spinal dysraphism, Stable angina, Staph Wound Infections, Staph Wound Infections and Methicillin Resistant Staphylococcus aureus, Stein-Leventhal Syndrome, Stroke, Swine flu, Syndrome X, Syphilis, Systemic Lupus Erythematosus, Systemic Scleroderma, Systemic Sclerosis, Testicular Cancer, Thalassemia, Thrombophilia, Thyroid Diseases, Toxic diffuse goiter, Travelers' Diseases, Trichomonas, Trisomy 21, Tuberculosis, Unstable angina, Urinary Tract Infection, Vasculitis, Venereal diseases, Vitamin B12 Deficiency, Vitamin B12 Deficiency and Folate Deficiency, Vitamin K Deficiency, Walking pneumonia, West Nile Virus, Wilson Disease, and Wound and Skin Infections.

Example health categories corresponding to family history include, but are not limited to: heart attack, coronary bypass surgery, Rheumatic or other heart disease, stroke, breast cancer, colon cancer, hip fracture, asthma, alzheimer's disease, high blood pressure, high blood cholesterol, and diabetes.

Example health categories corresponding to health interests include, but are not limited to any other health category described herein and/or various lifestyle categories (e.g., working out and meditation, among other examples).

Example health categories corresponding to genetics include, but are not limited to: disease risks and the various types thereof (e.g., Alzheimer's Disease, Psoriasis, Colorectal Cancer, Multiple Sclerosi, Primary Biliary Cirrhosis, Scleroderma (Limited Cutaneous Type), Esophageal Squamous Cell Carcinoma (ESCC), Stomach Cancer (Gastric Cardia Adenocarcinoma), Alopecia Areata, Bladder Cancer, Celiac Disease: Preliminary Research, Chronic Lymphocytic Leukemia, Glaucoma: Preliminary Research, Gout, Hodgkin Lymphoma, Keloid, Kidney Cancer, Paget's Disease of Bone, Primary Biliary Cirrhosis, Restless Legs Syndrome: Preliminary Research, Sarcoma, Cleft Lip and Cleft Palate, Developmental Dyslexia, Myeloproliferative Neoplasms, Sjögren's Syndrome, Breast Cancer, Venous Thromboembolism, Rheumatoid Arthritis, Restless Legs Syndrome, Age-related Macular Degeneration, Melanoma, Exfoliation Glaucoma, Ulcerative Colitis, Type 1 Diabetes, Celiac Disease, Crohn's Disease, Atrial Fibrillation: Preliminary Research, Bipolar Disorder: Preliminary Research, Breast Cancer Risk Modifiers, Dupuytren's Disease, Endometriosis, Gestational Diabetes, Hypothyroidism, Kidney Disease, Kidney Stones, Lou Gehrig's Disease (ALS), Nicotine Dependence, Osteoarthritis, Ovarian Cancer, Pancreatic cancer, Peripheral Arterial Disease, Polycystic Ovary Syndrome, Sarcoidosis, Melanoma: Preliminary Research, Thyroid Cancer, Uterine Fibroids, Back Pain, Creutzfeldt-Jakob Disease, Nonalcoholic Fatty Liver Disease, Obesity, Coronary Heart Disease, Diabetes, Atrial Fibrillation, Gallstones, Lung Cancer, Chronic Kidney Disease, Parkinson's Disease, Lupus (Systemic Lupus Erythematosus), Bipolar Disorder, Prostate Cancer, Abdominal Aortic Aneurysm, Ankylosing Spondylitis, Asthma, Atopic Dermatitis, Behcet's Disease. Brain Aneurysm, Chronic Obstructive Pulmonary Disease (COPD), Coronary Heart Disease, Follicular Lymphoma, Generalized Vitiligo, High Blood Pressure (Hypertension), Migraines, Progressive Supranuclear Palsy, Selective IgA Deficiency, Alcohol Dependence, Basal Cell Carcinoma, Cluster Headaches, Esophageal Cancer, Hay Fever (Allergic Rhinitis), Heart Rhythm Disorders (Arrhythmias), Larynx Cancer, Meningioma, Narcolepsy, Nasopharyngeal Carcinoma, Neuroblastoma, Oral and Throat Cancer, Otosclerosis, Parkinson's Disease, Schizophrenia, Scoliosis, Squamous Cell Carcinoma, Stomach Cancer, Stroke, Sudden Cardiac Arrest, Male Infertility, Testicular Cancer, Attention-Deficit Hyperactivity Disorder, Essential Tremor, Hashimoto's Thyroiditis, Hypertriglyceridemia, Neural Tube Defects, Placental Abruption, Preeclampsia, Tardive Dyskinesia, Intrahepatic Cholestasis of Pregnancy, Obsessive-Compulsive Disorder, and Tourette's Syndrome), carrier status (e.g., Alpha-1 Antitrypsin Deficiency, Agenesis of the Corpus Callosum with Peripheral Neuropathy (ACCPN), Autosomal Recessive Polycystic Kidney Disease, ARSACS, Bloom's Syndrome, BRCA Cancer Mutations, Canavan Disease, Congenital Disorder of Glycosylation Type 1a (PMM2-CDG), Cystic Fibrosis, D-Bifunctional Protein Deficiency, Dihydrolipoamide Dehydrogenase Deficiency, DPD Deficiency, Familial Dysautonomia, Factor XI Deficiency, Fanconi Anemia (FANCC-related), Familial Hypercholesterolemia Type B, Familial Hyperinsulinism (ABCC8-related), Familial Mediterranean Fever, G6PD Deficiency, Gaucher Disease, GRACILE Syndrome, Glycogen Storage Disease Type 1a, Glycogen Storage Disease Type 1b, Hemochromatosis, Primary Hyperoxaluria Type 2 (PH2), Hypertrophic Cardiomyopathy (MYBPC3 25 bp-deletion), LAMB3-related Junctional Epidermolysis Bullosa, Limb-girdle Muscular Dystrophy, Medium-Chain Acyl-CoA Dehydrogenase (MCAD) Deficiency, Maple Syrup Urine Disease Type 1B, Mucolipidosis IV, Neuronal Ceroid Lipofuscinosis (CLN5-related), Neuronal Ceroid Lipofuscinosis (PPT1-related), Niemann-Pick Disease Type A, Nijmegen Breakage Syndrome, Connexin 26-Related Sensorineural Hearing Loss, Pendred Syndrome, Phenylketonuria, Rhizomelic Chondrodysplasia Punctata Type 1 (RCDP1), Salla Disease, Sickle Cell Anemia & Malaria Resistance, Tay-Sachs Disease, Torsion Dystonia, and Zellweger Syndrome Spectrum), drug response (e.g., Abacavir Hypersensitivity, Clopidogrel (Plavix®) Efficacy, Fluorouracil Toxicity, Response to Hepatitis C Treatment, Pseudocholinesterase Deficiency, Warfarin (Coumadin®) Sensitivity, Caffeine Metabolism, Hepatitis C Treatment Side Effects, Metformin Response, Antidepressant Response, Beta-Blocker Response, Floxacillin Toxicity, Heroin Addiction, Lumiracoxib (Prexige®) Side Effects, Naltrexone Treatment Response, Postoperative Nausea and Vomiting (PONV), Response to Interferon Beta Therapy, and Statin Response), and traits (e.g., memory, response to exercise, etc.).

It should be understood that the various health categories described herein are provided for purposes of example and explanation only. Other examples of such health categories may exist, and the examples set forth herein should not be taken to be limiting.

Health category metrics corresponding to each category may be, or may be determined based on, commonly used referenced ranges, known medical decision tree algorithms, or custom made algorithms (e.g. generated via a Populational feedback loop of longitudinal patient data based on outcomes). A given health category metric may provide, for example, an indication of diagnostic status, disease progression, risk assessment, or a pharmacological response activity of a drug, among other examples. For example, labs related to cardiovascular disease (total cholesterol, ldl, hdl, triglycerides, Apolipoprotein B, urinary microalbumin, and myloperoxidase) may be categorized and a corresponding health category metric may be determined. In such a case, a qualitative risk-assessment health category metric of developing cardiovascular disease (e.g. High-risk) may be determined. Additionally, sub-metric(s) may also be determined for each category to further stratify the patient (e.g. near-term risk of have a cardiac event).

Notably, the one or more health-category metrics may be, or may be determined based on, a qualitative- or quantitative-based reference-range value. Alternatively, the one or more health-category metrics may be determined from a multiple test-driven medical algorithm decision tree value. Correspondingly, determining the set of health categories may involve sorting the laboratory test data based on a medical algorithm decision tree. Other examples of health-category metrics may exist.

In an implementation, the computing system may also determine, based on the set of health categories, at least one actionable-treatment choice. Then (perhaps in accordance with block 2508 below), the computing system may cause a visual indication of the determined at least one actionable-treatment choice to be displayed on the graphical display.

Determination of the at least one actionable-treatment choice may be performed after determination of the health categories and the corresponding health category metrics. The actionable-treatment choice may take the form of possible and/or medically validated treatment choices (e.g. pharmaceutical drugs, diet recommendations, life style, and supplements and vitamins, among other examples) from a medical treatment database. In the case of a health category involving type-2-diabetes, for example, the system may determine various actionable treatment choices to choose from which might include lifestyle options (e.g. light exercise and walking daily), dietary recommendations (e.g. plant-based diet or diabetes supportive meals/recipes), pharmaceutical drugs (metformin and Januvia), and/or supplements or vitamins (e.g. chromium). In an implementation, the system may present multiple actionable-treatments choices to the user (e.g. patient), and the user may be able to select a particular actionable-treatment choice. Other examples may exist as well.

Accordingly, in an embodiment, the computing system may receive treatment-selection data indicating a selection of one or more of the at least one actionable-treatment choices. The computing system may then select, based on the received treatment-selection data, a visual indication of the selected one or more of the at least one actionable-treatment choices. And then, the computing system (perhaps in accordance with block 2508 below) may cause the selected visual indication of the selected one or more of the at least one actionable-treatment choices to be displayed on the graphical display. Moreover, the visual indication of the selected one or more of the at least one actionable-treatment choices may be displayed relative to the visual depiction of the human body.

For purposes of example and explanation, in an implementation, the health data may be received by the computing system using a secure HL7 data stream. A parsing engine stored locally on, or remotely to, the computing system may take the health data (e.g., using a pearl implementation), sort the health data, and store the health data in a database (e.g. a database that is accessible via SQL statements or queries). More particularly, the health data may be sorted and stored into suitable tables. And the computing system may create new data or data sets based on the health data (e.g., using C++ and/or SQL query combination). And an extraction engine may include program code that is configured to extract data out of the database (e.g. using JASON, an intermediate coding language). The particular implementation, and aspects thereof described above is set forth only for purposes of example. The mentioned languages, computing systems, and components thereof should not be taken to be limiting. Those of skill in the art will appreciate that the present method may be implemented using a wide variety of programming techniques and/or computing systems.

b. Select Health Category for Visual Indication

At block 2504, server 106 selects, based on the respective one or more health-category metrics corresponding to the health categories in the set of health categories, at least one health category for visual indication.

As noted above, the variety of health categories that may have been determined in accordance with block 2502 above may be extensive. As such, at block 2504, the system may dynamically choose which of those health categories determined in accordance with block 2502 may ultimately be displayed to the user. In an implementation, however, the system may select each determined health category for visual indication.

In an implementation, the system may determine which health categories are relatively more pertinent and/or relevant to a patient's health, and may select those health categories for visual indication. For example, if the health category “cardiovascular disease” is determined at block 2502 along with a corresponding health metric that suggests the patient is near-risk for a cardiac event, this category would take priority over, for example, a prostate health category with a corresponding health metric that suggests the patient has a healthy prostate. (In that example, the lower priority healthy prostate health category may or may not be subsequently presented to the user.)

As another example, a health category may be selected for visual indication if it was previously abnormal, but is now healthy. Health categories may also be pre-selected to be of user-interest, and thus are always presented (e.g. if one has a family history of a disease related to a health category). As another example, the system may be have information previously inputted by the user, physician, electronic health record, etc., of a drug or treatment the user is taking related to a health-category. As such, a category would automatically be presented for the user to monitor the drug or treatments efficacy. Alternatively, if a user wishes to view pertinent information regarding their genetic code using the system (which may contain over 23,000 genes, and thousands of disease and risk-assessment correlations), the system may automatically present the most pertinent categories to the user. Other examples for selecting a health category may exist.

As shown in FIG. 27, health categories diabetes, heart disease, liver and kidney, and nutrition have been automatically selected due to their relevance to the user. On this particular health date, the user was classified as abnormal diabetes results, abnormal heart disease results, and abnormal liver and kidney results, with nutrition being presented because it was previously abnormal, but is now healthy. Other categories are intentionally not presented due to their healthy status.

The examples of selecting health categories discussed above are provided for purposes of example and explanation only and should not be taken to be limiting. Furthermore, in an implementation, the system may select all health categories for visual indication. Additionally or alternatively, health categories may be selectable by the user for visual indication.

c. Select Visual Indication of Selected Health Category

At block 2506, server 106 selects, based on at least one of (i) the selected at least one health category and (ii) the respective one or more health-category metrics corresponding to the selected at least one health category, a visual indication of the selected at least one health category.

Examples of the selection of particular visual indications are discussed below with respect to the Figures. It should be generally understood that the computing system may be arranged to select such visual indications, from among many alternative such visual indications. Moreover, as discussed further below with respect to block 2508, the computing system may ultimately display the selected visual indication. As a general matter, the selected visual indication may include any of those visual indication described above with respect to method 400 of FIG. 4, among other visual indications, some examples of which are discussed below. The example visual indications provided herein are provided for purposes of example and explanation only and should not be taken to be limiting.

The visual indication that is selected may include (or otherwise specify) any suitable parameter, characteristic, or other feature of a graphical user interface element. For instance, the visual indication may specify a layout, order, size, or other positional other positional characteristic. The visual indication may also specify other graphical elements such as color. Moreover the visual indication may specify a particular graphic or animation, from among a database of available graphics or animations. Other examples of visual indications may exist.

As a general matter, the visual indication may help convey health information that the user may be interested in, and may do so in a manner that supports the user's understanding of the health information. As such, the selected visual indication of the at least selected one health category may include at least one of a diagnosis associated with the selected at least one health category, a risk-assessment and/or prognosis associated with the selected at least one health category, and a therapeutic response to a drug associated with the selected at least one health category. Other examples may exist.

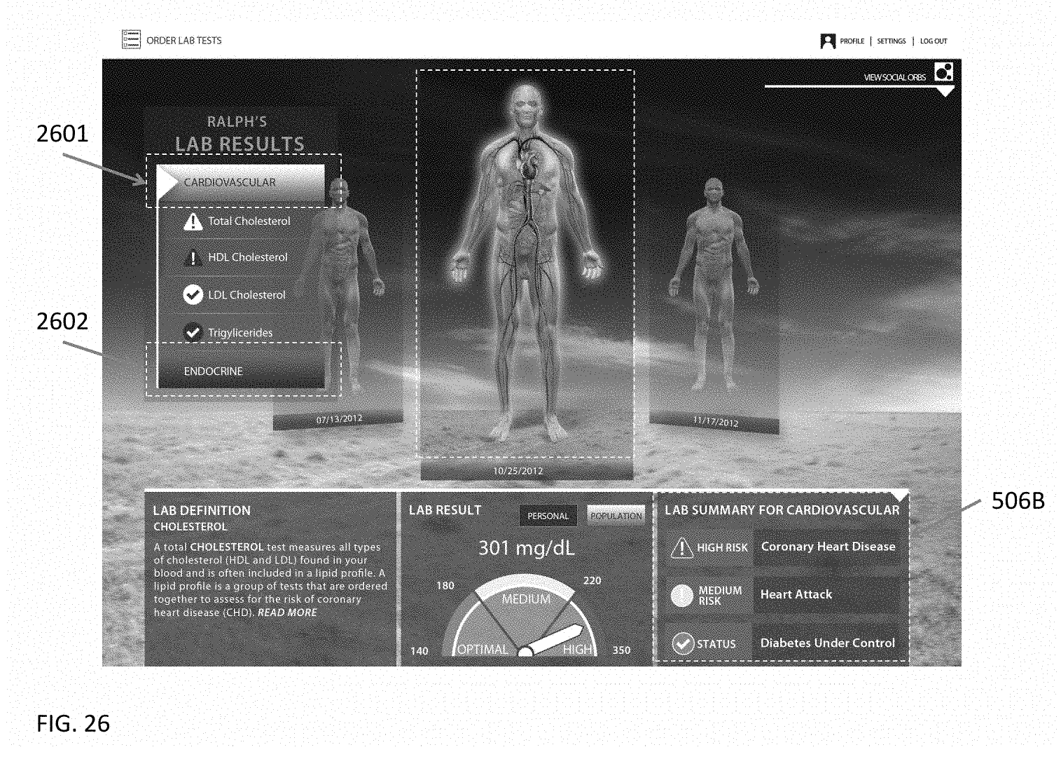

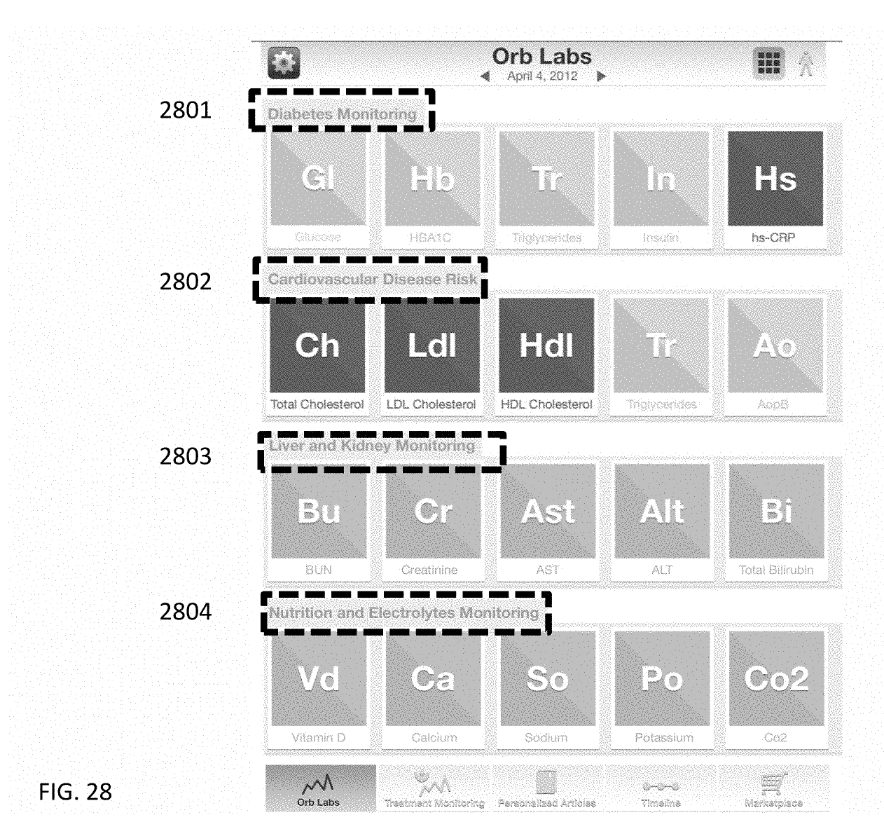

In FIG. 26, for example, a visual indication of two health categories are presented: cardiovascular and endocrine (2601 and 2602). Further, visual indications of various health category metrics for the cardiovascular health category are presented separately (2603) as high-risk of cardiovascular disease, medium risk of a heart attack, and diabetes under control. In a more simplified interface, FIG. 27 presents health categories diabetes (2701), heart disease (2702), liver and kidney (2703), and nutrition (2704). Unlike FIG. 26, in FIG. 27 the health categories are presented in combination and visualized via particular colors such as, in an example, green (nutrition), yellow (diabetes, liver and kidney), and red (heart disease) colors in order of their relative severity or risk-assessment. Of note, new FIG. 28 presents, in a similar fashion, laboratory tests that are used to determine health-category metrics for each of the health categories in FIG. 27 (in this example, quantitative lab values are hidden and replaced with a corresponding color scheme).

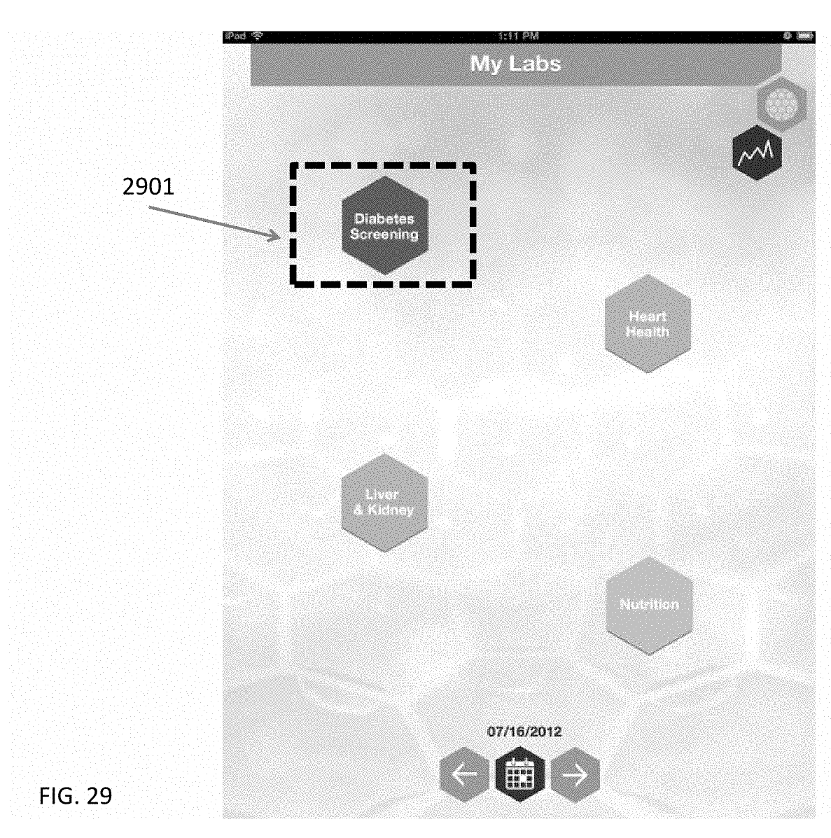

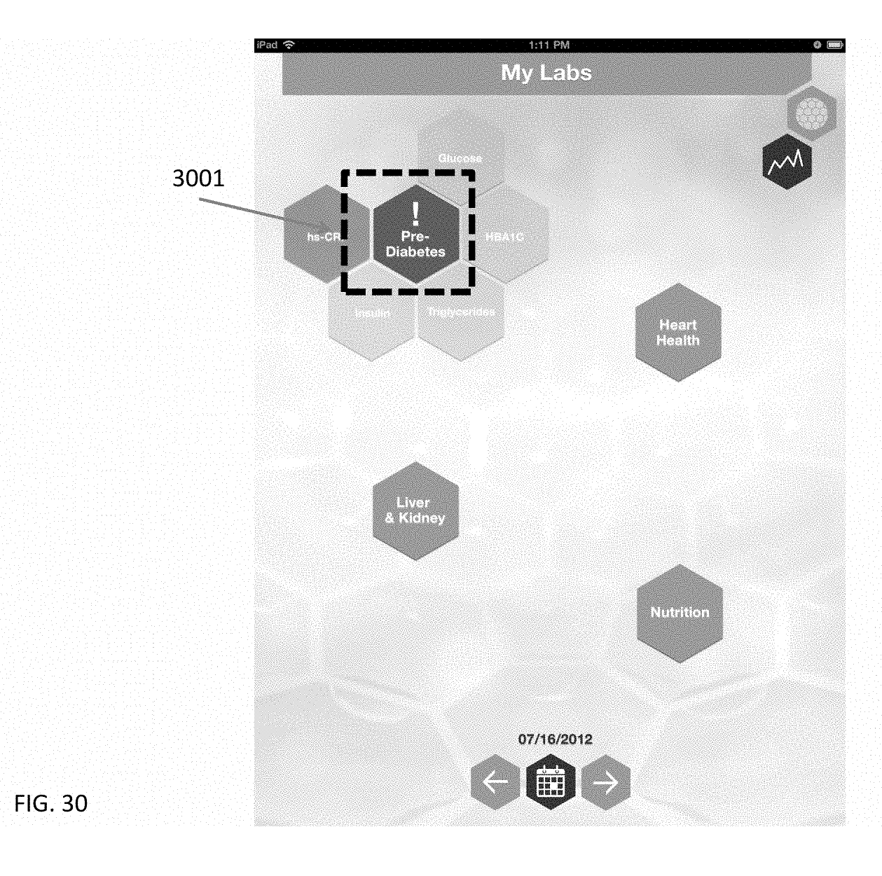

In FIG. 29, a similar approach to creation of health categories and health-category metrics is used to select visual indications. For example, the results from the diabetes screening (2901), were selected for visual indication as a particular color such as, in an example, red, indicating abnormal results and a diabetes presence. Details pertaining to this health category can be further specified/classified as seen in FIG. 30. After a user selects (2901) in FIG. 29, this health category further classified to be Pre-Diabetes (3001), with visual indications of relevant labs that were involved in generating the health category and corresponding health category metrics also appearing.

In an implementation, therefore, selecting the at least one health category for visual indication comprises selecting the at least one health category based on a respective criticality score corresponding to the at least one health category. For instance, in the example discussed above with respect to FIG. 29, the results from the diabetes screening indicated abnormal results. As a result, the system may associate a high criticality score with the diabetes health category, and therefore may select a red visual indication to display with respect to that health category. On the other hand, in the example discussed above with respect to FIG. 27, the nutrition health category is displayed because it was previously unhealthy, but is now healthy. As a result, the system may associate a low criticality score with the nutrition health category, and therefore may select a green visual indication to display with respect to that health category.

The criticality score may additionally, or alternatively, be used to provide an indication of relative importance of a given health category when compared to another health category. For instance, a health category associated with heart health may intrinsically possess a criticality score that is higher than prostate health, and therefore the heart health category may be displayed more commonly (or in a more prominent manner) than the prostate health category.

Of note, the computing system may ultimately display, relative to a human body (e.g., adjacent to, on top of, and/or within the human body), visual indications of a diagnosed disease, a projected future health event, the progression of a disease, and/or how a drug is working and affecting one's biology, among other examples. Each such visual indication may be selected based on the health categories and/or health-category metrics determined in accordance with the blocks described above. More examples may exist.

In an implementation where the visual indication is on top of or within the visual indication of the human body, the computing system may determine, based on the received user health data, a set of physiological features. The computing system may then select at least one physiological feature from the set of physiological features for visual indication. Further, the computing system may select a visual indication of the selected at least on physiological feature. And then (perhaps in accordance with block 2508 discussed further below), the computing system may cause the selected visual indication of the selected at least on physiological feature to be displayed on a graphical display, where the selected at least on physiological feature is displayed relative to the visual depiction of a human body.

FIGS. 26 and 27 each present a visual depiction of a human body including visual depictions of physiological features selected based on health categories and corresponding health-category metrics. In FIG. 26, the human body's cardiovascular system is highlighted and animated due to the biologically associated lab results. This body interface shown in FIG. 26 may be manipulated and zoomed into (e.g. into a coronary heart vein). In essence, the human body, or user “avatar,” generally serves to simulate a disease, prognosis, or response to a therapeutic.

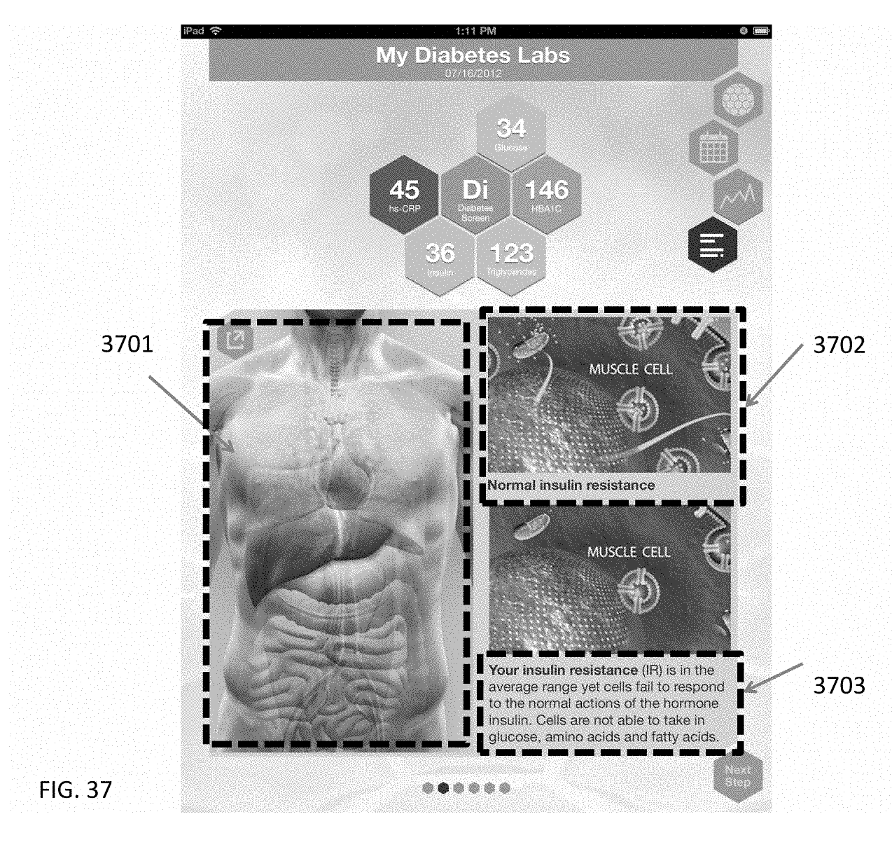

In FIG. 27, visual depictions are provided relative to the human body so as to simulate different affected organ systems known to become aberrant with type-2-diabetes. For instance, the liver is highlighted/animated due to its known increased production of cholesterol associated with diabetes and the down-stream affects that can lead to cardiovascular disease. Body visualizations/animations arising from category metrics (healthy processes, disease processes, disease progression, risk-assessment via a predicted future health event, response to a therapeutic drug, etc.) may exist in a multitude of possibilities, including systematic body presentations (e.g. the relationship across the entire endocrine system related to diabetes), macroscopic organ-system views that are dynamically highlighted or animated (e.g. the pancreas and the Islets of Langerhans), microscopic views into the pathophysiology of health and disease (e.g. into the blood stream observing glucose and A1C), inside cells (glucose uptake), inside a cellular nucleus (gene's known to increase one's predisposition for diabetes). Additional presentations also exist wherein the user-interface may present a “magazine” or “digital reader” user-interface (FIG. 37) containing the human body (3701), microscopic pathophysiology view (3702), and supporting text to guide the reader (3703), side-by-side.

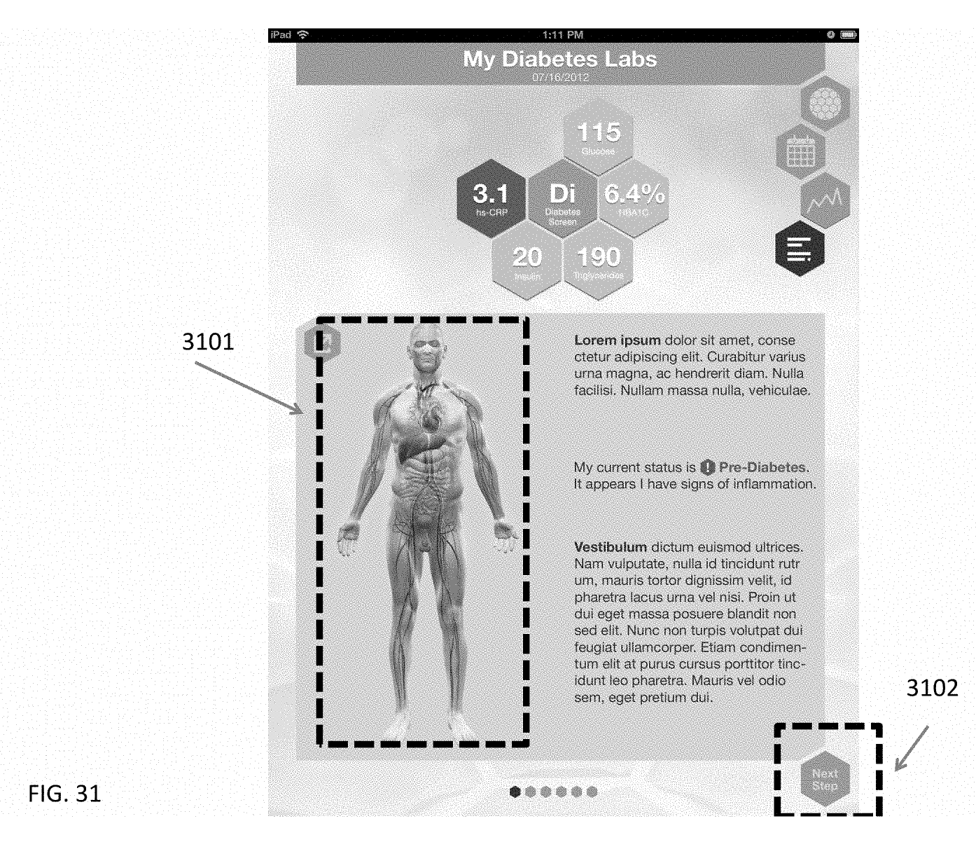

Generally, the visual indication of each health category may include a visual indication of the health-category metrics corresponding to the health category. The visual indication of the health-category metric may take any suitable form such as a color (as described above), a number, or some other suitable form. As described above, FIGS. 27 and 28 each present health-category metrics corresponding to health categories via the use of colors. Red may correspond to high risk/disease presence, yellow may correspond to medium/low risk, and green may correspond to relatively good health. Other examples may exist. FIG. 31 presents an alternative screen to visualize the body in the context of diabetes (3101).

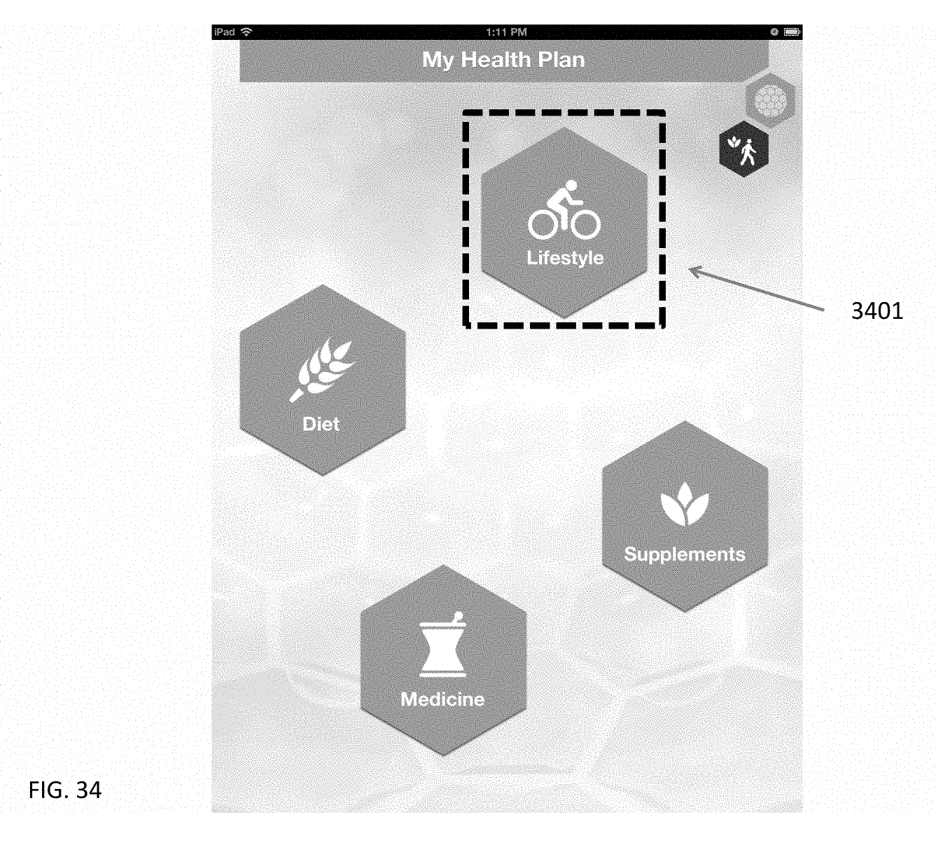

As noted above, the system may select for display an actionable-treatment choice. In an implementation, the visual indication of the selected one or more of the at least one actionable-treatment choices may include a visual indication of how the at least one actionable-treatment choices affects the body. For instance, the system may allow the user to select actionable treatment choices as well. In the interface depicted in FIG. 31, the user can simply select button (3102) to begin exploring known treatment solutions for diabetes. FIG. 34 presents a user interface to guide the user to select from known lifestyle solutions (3401), diet solutions, medicine (drugs), and supplement solutions. More examples may exist.

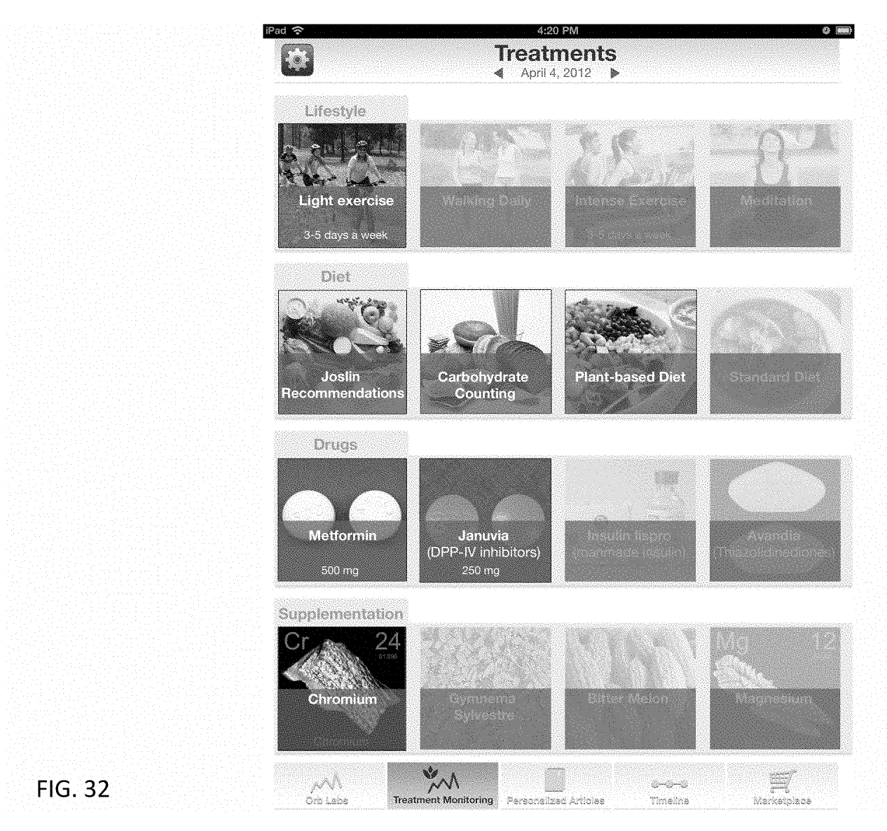

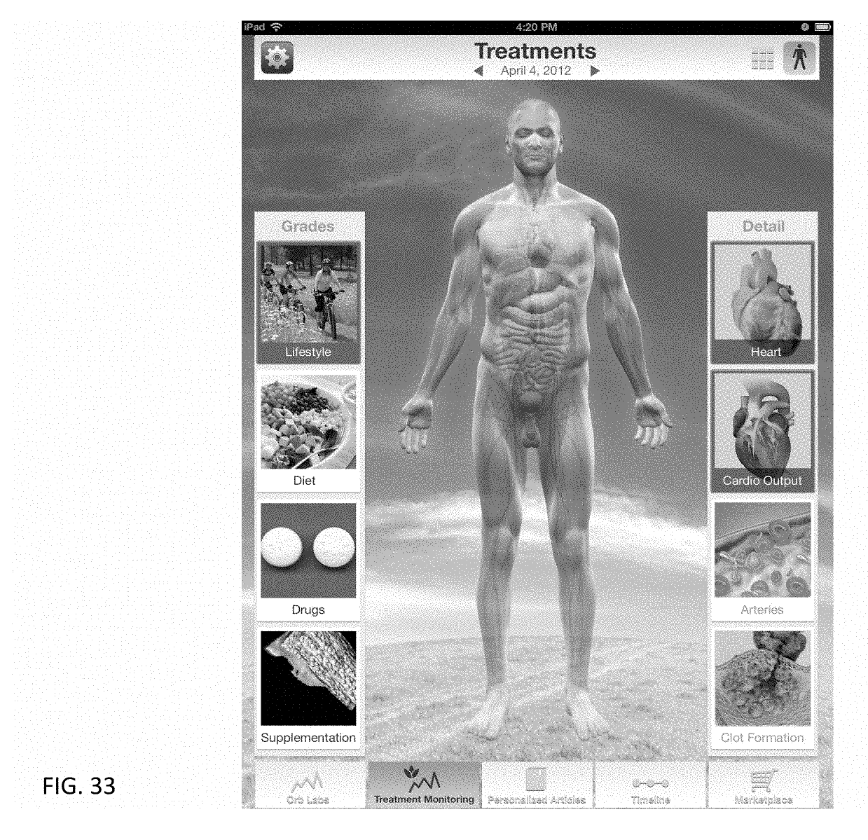

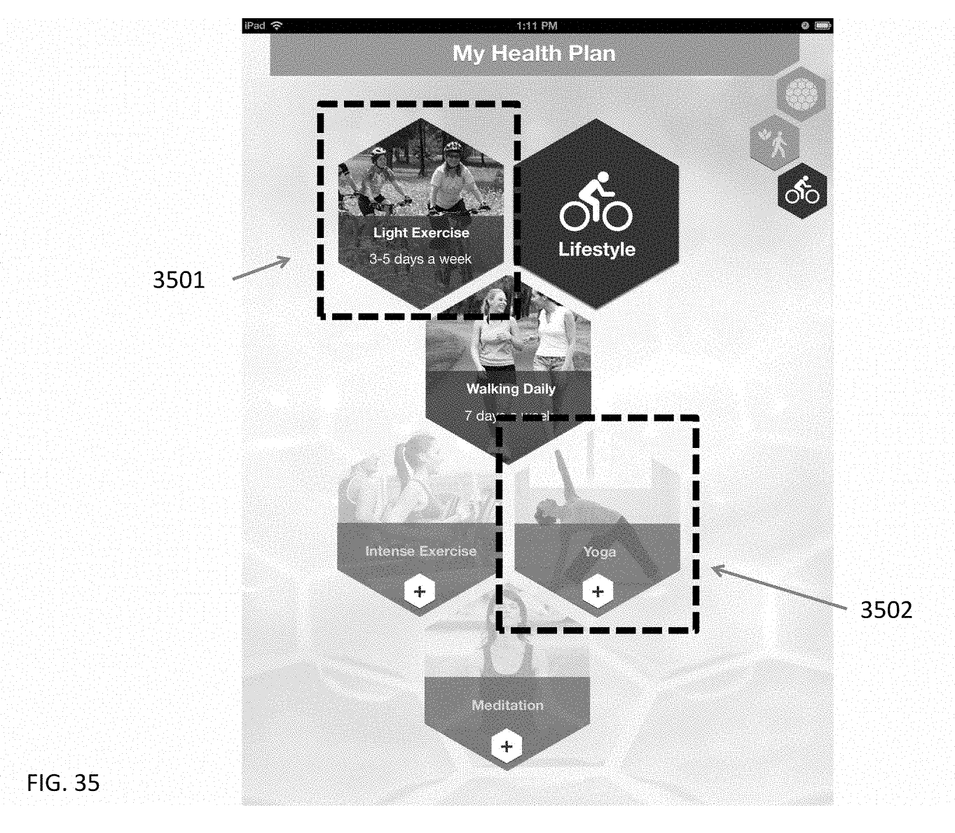

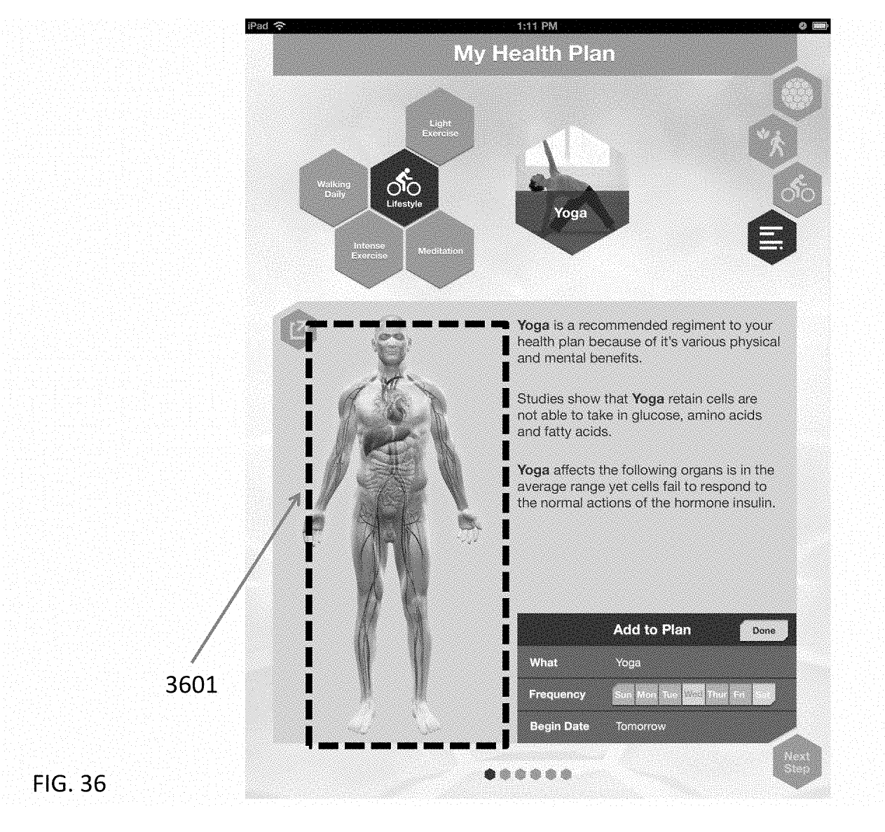

In the example shown in FIG. 35, for example, the user previously selected a “lifestyle solutions” (3401) buttons to view currently used lifestyle solutions (that were previously identified by the user, e.g. 3501), as well as additional alternative lifestyle solutions known to successfully support diabetes care/management (3502). Then, as shown in FIG. 36, the user may make a selection, a new treatment or existing treatment option (such as one of “light exercise,” “meditation,” and/or “intense exercise,” among other examples) and then visualize how this treatment choice supports, affects, or treats the body. Alternative example user interfaces that include the presentation of various actionable-treatment choices and associated information are presented in FIGS. 32 and 33.

d. Cause Visual Indication to be Displayed

At block 2508, server 106 causes the selected visual indication of the selected at least one health category to be displayed on a graphical display. As noted above, the visual indication of the selected at least one health category may be displayed relative to a visual depiction of a human body. The visual indication that is displayed may include any of those visual indications described above with respect to block 2508.

The visual indication may be displayed using, at least in part, one or more of Objective C, C++, Java, or Unity programming language. Those of skill in the art will understand that many techniques exist to cause the visual indication to be displayed. The examples provided herein should not be taken to be limiting.

Those of skill in the art will appreciate that the use of visual indications described herein displayed relative to a human body to visually depict health categories and health-category metrics (based, in an implementation, on laboratory test data) may provide the user (such as a patient or consumer, among other examples) with an interface that “personifies” what is typically raw, numerical, uncategorized, data indicative of disease presence, disease progression, risk-assessment or prediction of future-health event, or response to a therapeutic drug, among other examples. Such an interface may be particularly beneficial, in some implementations, to individuals not professionally trained in the medical field. Such personification of data enables users to more easily understand health data.

### 4. Conclusion

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

I claim:

1. A system comprising:
a processor;
a non-transitory computer readable medium; and
program instructions stored on the non-transitory computer readable medium and executable by the processor to:
determine, based on received user health data, (i) a set of health categories and (ii) for each health category in the set of health categories, one or more respective health-category metrics corresponding to the health category;
select, based on the respective one or more health-category metrics corresponding to the health categories in the set of health categories, at least one health category for visual indication;
select, based on at least one of (i) the selected at least one health category and (ii) the respective one or more health-category metrics corresponding to the selected at least one health category, a visual indication of the selected at least one health category; and
cause the selected visual indication of the selected at least one health category to be displayed on a graphical display, wherein the visual indication of the selected at least one health category is displayed relative to a visual depiction of a human body.

2. The system of claim 1, further comprising program instructions stored on the non-transitory computer readable medium and executable by the processor to:
cause a visual indication of each health category in the set of health categories to be displayed on the graphical display.

3. The system of claim 2, wherein the visual indication of each health category comprises a visual indication of the health-category metrics corresponding to the health category.

4. The system of claim 1, further comprising program instructions stored on the non-transitory computer readable medium and executable by the processor to:
determine, based on the set of health categories, at least one actionable-treatment choice; and
cause a visual indication of the determined at least one actionable-treatment choice to be displayed on the graphical display.

5. The system of claim 4, further comprising program instructions stored on the non-transitory computer readable medium and executable by the processor to:
receive treatment-selection data indicating a selection of one or more of the at least one actionable-treatment choices;
select, based on the received treatment-selection data, a visual indication of the selected one or more of the at least one actionable-treatment choices; and
cause the selected visual indication of the selected one or more of the at least one actionable-treatment choices to be displayed on the graphical display, wherein the visual indication of the selected one or more of the at least one actionable-treatment choices is displayed relative to the visual depiction of the human body.

6. The system of claim 5, wherein the visual indication of the selected one or more of the at least one actionable-treatment choices comprises a visual indication of how the at least one actionable-treatment choices affects the body.

7. The system of claim 1, wherein each respective health category in the set of health categories is one of cardiovascular disease, diabetes, mental health, nutrition, cancer, kidney disease, alzheimer's disease, stroke, and obesity.

8. The system of claim 1, wherein the one or more health-category metrics is one of a (a) a qualitative value or (b) a quantitative value.

9. The system of claim 1, wherein selecting the at least one health category for visual indication comprises selecting the at least one health category based on a respective criticality score corresponding to the at least one health category.

10. The system of claim 1, further comprising program instructions stored on the non-transitory computer readable medium and executable by the processor to:
determine, based on received user health data, a set of physiological features;
select at least one physiological feature from the set of physiological features for visual indication;
select a visual indication of the selected at least on physiological feature; and
cause the selected visual indication of the selected at least on physiological feature to be displayed on a graphical display, wherein the selected at least on physiological feature is displayed relative to a visual depiction of a human body.

11. The system of claim 1, wherein the selected visual indication of the at least selected one health category comprises at least one of a diagnosis associated with the selected at least one health category, a risk-assessment and/or prognosis associated with the selected at least one health category, and a therapeutic response to a drug associated with the selected at least one health category.

12. The system of claim 1, wherein the user health data is laboratory test data.

13. The system of claim 11, wherein determining the set of health categories comprises sorting the laboratory test data based on a medical algorithm decision tree.

14. A non-transitory computer readable medium having instructions stored thereon, the instructions comprising:
instructions for determining, based on received user health data, (i) a set of health categories and (ii) for each health category in the set of health categories, one or more respective health-category metrics corresponding to the health category;
instructions for selecting, based on the respective one or more health-category metrics corresponding to the health categories in the set of health categories, at least one health category for visual indication;
instructions for selecting, based on at least one of (i) the selected at least one health category and (ii) the respective one or more health-category metrics corresponding to the selected at least one health category, a visual indication of the selected at least one health category; and
instructions for causing the selected visual indication of the selected at least one health category to be displayed on a graphical display, wherein the visual indication of the selected at least one health category is displayed relative to a visual depiction of a human body.

15. The non-transitory computer readable medium of claim 14, the instructions further comprising:
instructions for causing a visual indication of each health category in the set of health categories to be displayed on the graphical display.

16. A computer-implemented method comprising:
determining, based on received user health data, (i) a set of health categories and (ii) for each health category in the set of health categories, one or more respective health-category metrics corresponding to the health category;
selecting, based on the respective one or more health-category metrics corresponding to the health categories in the set of health categories, at least one health category for visual indication;
selecting, based on at least one of (i) the selected at least one health category and (ii) the respective one or more health-category metrics corresponding to the selected at least one health category, a visual indication of the selected at least one health category; and
causing the selected visual indication of the selected at least one health category to be displayed on a graphical display, wherein the visual indication of the selected at least one health category is displayed relative to a visual depiction of a human body.

17. The computer-implemented method of claim 14, further comprising:
causing a visual indication of each health category in the set of health categories to be displayed on the graphical display.

18. A system comprising:
a processor;
a non-transitory computer readable medium; and
program instructions stored on the non-transitory computer readable medium and executable by the processor to:
receive test data indicating laboratory-test information corresponding to at least one patient-test date;
receive, via a user interface, (i) analysis-type data indicating an analysis type and (ii) patient-history data indicating a particular patient-test date;
determine a test result based on at least the (i) received test data, (ii) the analysis-type data, and (iii) the patient-history data; and
cause a visual depiction of the test result relative to a visual depiction of a human body to be displayed on a graphical display.

19. The system of claim 18, further comprising program instructions stored on the non-transitory computer readable medium and executable by the processor to:
determine, based on the received test data, (i) a set of health categories and (ii) for each health category in the set of health categories, one or more respective health-category metrics corresponding to the health category;
select, based on the respective one or more health-category metrics corresponding to the health categories in the set of health categories, at least one health category for visual indication;
select, based on at least one of (i) the selected at least one health category and (ii) the respective one or more health-category metrics corresponding to the selected at least one health category, a visual indication of the selected at least one health category; and
cause the selected visual indication of the selected at least one health category to be displayed on a graphical display, wherein the visual indication of the selected at least one health category is displayed relative to a visual depiction of a human body.

20. The system of claim 19, further comprising program instructions stored on the non-transitory computer readable medium and executable by the processor to:
cause a visual indication of each health category in the set of health categories to be di splayed on the graphical display.
